SEQUENCE LISTING (1 sequence)

SEQ ID NO: 1 - protein, 42 aa, Homo sapiens [sequence omitted]

(19) United States
(12) Patent Application Publication
(10) Pub. No.: US 2023/0028659 A1
(43) Pub. Date: Jan. 26, 2023

(54) THERAPEUTIC COMPOSITIONS AND METHODS FOR PREVENTION AND TREATMENT OF DIASTOLIC DYSFUNCTION

(71) Applicant: AMBETEX PTY LTD, Geelong West (AU)

(72) Inventor: Sean MCGEE, Geelong West (AU)

(73) Assignee: AMBETEX PTY LTD, Geelong West (AU)

(21) Appl. No.: 17/784,593

(22) PCT Filed: Dec. 9, 2020
(86) PCT No.: PCT/AU2020/051350

§ 371 Date: Jun. 10, 2022

(30) Foreign Application Priority Data
Dec. 11, 2019 (AU) 2019904686

(51) Int. Cl. A61K 31/4025; A61P 9/00; A61K 9/00

(52) U.S. Cl. CPC A61K 31/4025; A61P 9/00; A61K 9/0019

(57) ABSTRACT

Methods for preventing or treating diastolic dysfunction in an individual comprising administering to an individual in need of said prevention or treatment a therapeutically effective amount of a mGluR5 negative allosteric modulator, compositions comprising a mGluR5 negative allosteric modulator for use in treatment of diastolic dysfunction and pharmaceutical compositions comprising same.

### FIELD OF THE INVENTION

The invention relates to diastolic dysfunction and related conditions and to mGluR5 negative allosteric modulators and therapeutic uses of same.

### BACKGROUND OF THE INVENTION

Diastole is the part of the cardiac cycle that includes the isovolumetric relaxation phase and the filling phases and has passive and active components. The filling of the left ventricle (LV) is divided into rapid filling during early diastole, diastasis, and a rapid filling phase late in diastole that corresponds with atrial contraction.

LV relaxation, an essential characteristic of normal diastole, is an energy-dependent process. In particular, adenosine triphosphate (ATP) is required to pump free myoplasmic calcium back into the sarcoplasmic reticulum, to extrude the calcium ions which enter the cell during the plateau phase of the action potential, and to extrude sodium that has been exchanged for calcium via sodium/potassium ATPase and an ATP-dependent calcium pump. Thus, when ATP production is limited, for example where there has been an impairment in the cardiac uptake of glucose, and/or impairments in mitochondrial metabolism, this may result in a slower rate of isovolumic relaxation and reduced distensibility of the LV.

Left ventricular diastolic dysfunction (LVDD) is a preclinical condition defined as the inability of the LV to fill an adequate end diastolic volume (preload volume) at an acceptable pressure.

LVDD is generally a consequence of abnormalities during diastole. The aforementioned impaired LV relaxation, high filling pressure, and increased LV operating stiffness are underlying mechanisms in LVDD. Cardiac impairments that represent LVDD include reduced E:A ratio and increased deceleration time. These impairments can lead to cardiac hypertrophy and associated cardiomyopathy, and heart failure.

Epidemiological evidence suggests there is a latent phase in which diastolic dysfunction is present and progresses in severity before the symptoms of heart failure arise. Asymptomatic mild LVDD is found in 21%, and moderate or severe diastolic dysfunction is present in 7% of the population.

In early diastolic dysfunction, elevated LV stiffness is associated with diastolic filling abnormalities and normal exercise tolerance. Asymptomatic diastolic dysfunction may be present for significant periods before it develops into a symptomatic clinical event. When the disease progresses, pulmonary pressures increase abnormally during exercise, producing reduced exercise tolerance. When filling pressures increase further, clinical signs of heart failure appear. In a significant number of cases of diastolic heart failure, patients have atrial fibrillation at the time of diagnosis, suggesting an association and a possible common pathogenesis. With atrial fibrillation, diastolic dysfunction may rapidly lead to overt diastolic heart failure.

The asymptomatic phase of diastolic dysfunction represents a potential time to intervene to prevent symptomatic heart failure. Suggesting the success of possible interventions, a mortality benefit has been observed in those whose diastolic dysfunction improved compared with those whose diastolic dysfunction remained the same or worsened.

Patients with LVDD are generally older, more often female, and have a high prevalence of CVD and other morbid conditions, such as obesity, metabolic syndrome, diabetes mellitus type 2, salt-sensitive hypertension, atrial fibrillation, COPD, anemia, and/or renal dysfunction.

LVDD may lead to heart failure with preserved ejection fraction (HFPeF). In HFPeF, normal ejection fraction is observed, but only at the expense of increased LV filling pressure. HFPeF is sometimes referred to as ‘diastolic heart failure’ or ‘backward heart failure’.

LVDD is an important precursor to many different cardiovascular diseases. It represents the dominant mechanism (⅔ of patients) in the development of HFPeF. HFPeF shows a rising prevalence in the older population. By 2020, more than 8% of people over 65 are estimated to have HFPEF and is associated with a poor prognosis.

To date, there are no specific treatments for diastolic dysfunction to selectively enhance myocardial relaxation. Moreover, no drug has been developed to improve long-term outcomes for diastolic heart failure.

Ufer M. et al 2016 Clin. Therap. 38: 2589-2597 discusses a conduct of thorough QTc study involving intra venous infusion of mavoglurant.

US 2008/0025966 A1 (Currie) discusses chymotrypsin inhibitors for treating various disorders including IBD and other gastrointestinal disorders and conditions.

There is a need for methods and compositions for providing improvements in the treatment or prevention of diastolic dysfunction.

### SUMMARY OF THE INVENTION

The invention relates to methods of treating, preventing, or ameliorating diastolic dysfunction or conditions associated with, or arising from same, and to pharmaceutical compositions and kits comprising mGluR5 negative allosteric modulators (mGluR5 NAM) in an individual for treating or preventing diastolic dysfunction or conditions associated with, or arising from same.

The invention provides a method for preventing or treating diastolic dysfunction or condition associated with same in an individual comprising providing a therapeutically effective amount of a mGluR5 NAM in an individual.

The invention further provides a composition comprising a therapeutically effective amount of a mGluR5 NAM for use in preventing or treating diastolic dysfunction or condition associated with same in an individual. Preferably the individual has an elevated amount of Aβ42 in the individual's plasma.

The invention further provides a use of a composition comprising a mGluR5 NAM in the manufacture of a medicament for preventing or treating diastolic dysfunction or condition associated with same.

The invention further provides a method for preventing or treating diastolic dysfunction or condition associated with same in an individual comprising:
assessing, or having assessed a sample, preferably a plasma sample obtained from an individual for whom diastolic dysfunction is to be prevented or treated to determine the amount of Aβ42 in the sample; and
where the individual has an amount of Aβ42 that is greater than that observed in a control describing the amount of Aβ42 in an individual who does not develop, or does not have diastolic dysfunction;
   providing a mGluR5 NAM to the individual;
      thereby preventing or treating diastolic dysfunction or condition associated with same in the individual.

The invention further provides a kit comprising:
a mGluR5 NAM or pharmaceutical composition comprising same;
written instructions for use of the kit in an enumerated embodiment described below.

Various (enumerated) embodiments of the present invention are described herein. It will be recognized that features specified in each embodiment may be combined with other specified features to provide further embodiments of the present disclosure.

Embodiment 1: A method for preventing or treating diastolic dysfunction in an individual, preferably an obese, or pre-diabetic, or diabetic or elderly individual, more preferably an obese individual, or an individual having an elevated amount of Aβ42, more preferably an elevated plasma amount of Aβ42 comprising administering a therapeutically effective amount of a mGluR5 NAM to the individual, preferably wherein the mGluR5 NAM is selected from Table 1 or Table 2, preferably wherein the mGluR5 NAM is compound 1, 2 or 3 described herein.

Embodiment 2: A method for preventing or treating heart failure, more preferably HFpEF in an individual, preferably an obese, or pre-diabetic, or diabetic or elderly individual, more preferably an obese individual, or an individual having an elevated amount of Aβ42, more preferably an elevated plasma amount of Aβ42 comprising administering a therapeutically effective amount of a mGluR5 NAM to the individual, preferably wherein the mGluR5 NAM is selected from Table 1 or Table 2, preferably wherein the mGluR5 NAM is compound 1, 2 or 3 described herein.

Embodiment 3: A method for preventing or treating concentric hypertrophy in an individual, preferably an obese, or pre-diabetic, or diabetic or elderly individual, more preferably an obese individual, or an individual having an elevated amount of Aβ42, more preferably an elevated plasma amount of Aβ42 comprising administering a therapeutically effective amount of a mGluR5 NAM to the individual, preferably wherein the mGluR5 NAM is selected from Table 1 or Table 2, preferably wherein the mGluR5 NAM is compound 1, 2 or 3 described herein.

Embodiment 4: A method for preserving or decreasing left ventricle deceleration time in an individual, preferably in an obese, or pre-diabetic, or diabetic or elderly individual, more preferably an obese individual, or an individual having an elevated amount of Aβ42, more preferably an elevated plasma amount of Aβ42 comprising administering a therapeutically effective amount of a mGluR5 NAM to the individual, preferably wherein the mGluR5 NAM is selected from Table 1 or Table 2, preferably wherein the mGluR5 NAM is compound 1, 2 or 3 described herein.

Embodiment 5: A method for preserving or preventing intra-ventricular septal thickening in an individual, preferably in an obese, or pre-diabetic, or diabetic or elderly individual, more preferably an obese individual, or an individual having an elevated amount of Aβ42, more preferably an elevated plasma amount of Aβ42 comprising administering a therapeutically effective amount of a mGluR5 NAM to the individual, preferably wherein the mGluR5 NAM is selected from Table 1 or Table 2, preferably wherein the mGluR5 NAM is compound 1, 2 or 3 described herein.

Embodiment 6: A method for preserving or preventing an increase in left ventricular mass in an individual, preferably in an obese, or pre-diabetic, or diabetic or elderly individual, more preferably an obese individual, or an individual having an elevated amount of Aβ42, more preferably an elevated plasma amount of Aβ42 comprising administering a therapeutically effective amount of a mGluR5 NAM to the individual, preferably wherein the mGluR5 NAM is selected from Table 1 or Table 2, preferably wherein the mGluR5 NAM is compound 1, 2 or 3 described herein.

Embodiment 7: A method for preventing or treating cardiomyopathy, more preferably diabetic cardiomyopathy, or hypertrophic cardiomyopathy, or ischemic cardiomyopathy, or hypertensive cardiomyopathy in an individual, preferably an obese, or pre-diabetic, or diabetic or elderly individual, more preferably an obese individual, or an individual having an elevated amount of Aβ42, more preferably an elevated plasma amount of Aβ42 comprising administering a therapeutically effective amount of a mGluR5 NAM to the individual, preferably wherein the mGluR5 NAM is selected from Table 1 or Table 2, preferably wherein the mGluR5 NAM is compound 1, 2 or 3 described herein.

Embodiment 8: A method for preventing the reduction of cardiac glucose uptake, or for preventing the accumulation cardiac tri-acyl glycerol in an individual, preferably an obese, or pre-diabetic, or diabetic or elderly individual, more preferably an obese individual, or an individual having an elevated amount of Aβ42, more preferably an elevated plasma amount of Aβ42 comprising administering a therapeutically effective amount of a mGluR5 NAM, wherein the mGluR5 NAM is selected from Table 1 or Table 2, preferably wherein the mGluR5 NAM is compound 1, 2 or 3 described herein.

Embodiment 9: A method for preventing or treating obesity-associated cardiomyopathy in an individual, more preferably in an individual having an elevated amount of Aβ42, more preferably an elevated amount of plasma Aβ42 comprising administering a therapeutically effective amount of a mGluR5 NAM, wherein the mGluR5 NAM is selected from Table 1 or Table 2, preferably wherein the mGluR5 NAM is compound 1, 2 or 3 described herein.

Embodiment 10: A method for preventing a reduction of the peak velocity of blood flow across the mitral valve during early diastolic filling (herein Peak E wave) in an individual, preferably an obese, or pre-diabetic, or diabetic or elderly individual, more preferably an obese individual, or an individual having an elevated amount of Aβ42, more preferably an elevated plasma amount of Aβ42 comprising administering a therapeutically effective amount of a mGluR5 NAM, wherein the mGluR5 NAM is selected from Table 1 or Table 2, preferably wherein the mGluR5 NAM is compound 1, 2 or 3 described herein.

Embodiment 11: A method for preventing a reduction in cardiac performance in an individual, preferably an obese, or pre-diabetic, or diabetic or elderly individual, more preferably an obese individual, or an individual having an elevated amount of Aβ42, more preferably an elevated plasma amount of Aβ42 comprising administering a therapeutically effective amount of a mGluR5 NAM, wherein the mGluR5 NAM is selected from Table 1 or Table 2, preferably wherein the mGluR5 NAM is compound 1, 2 or 3 described herein.

Embodiment 12: A composition for use in preventing or treating diastolic dysfunction in an individual, preferably an obese, or pre-diabetic, or diabetic or elderly individual, more preferably an obese individual, or an individual having an elevated amount of Aβ42, more preferably an elevated plasma amount of Aβ42 comprising a therapeutically effective amount of a mGluR5 NAM, wherein the mGluR5 NAM is selected from Table 1 or Table 2, preferably wherein the mGluR5 NAM is compound 1, 2 or 3 described herein.

Embodiment 13: A composition for use in preventing or treating heart failure, more preferably HFpEF in an individual, preferably an obese, or pre-diabetic, or diabetic or elderly individual, more preferably an obese individual, or an individual having an elevated amount of Aβ42, more preferably an elevated plasma amount of Aβ42 comprising a therapeutically effective amount of a mGluR5 NAM, preferably wherein the mGluR5 NAM is selected from Table 1 or Table 2, preferably wherein the mGluR5 NAM is compound 1, 2 or 3 described herein.

Embodiment 14: A composition for use in preventing or treating concentric hypertrophy in an individual, preferably an obese, or pre-diabetic, or diabetic or elderly individual, more preferably an obese individual, or an individual having an elevated amount of Aβ42, more preferably an elevated plasma amount of Aβ42 comprising a therapeutically effective amount of a mGluR5 NAM, preferably wherein the mGluR5 NAM is selected from Table 1 or Table 2, preferably wherein the mGluR5 NAM is compound 1, 2 or 3 described herein.

Embodiment 15: A composition for use in preserving or decreasing left ventricle deceleration time in an individual, preferably an obese, or pre-diabetic, or diabetic or elderly individual, more preferably an obese individual, or an individual having an elevated amount of Aβ42, more preferably an elevated plasma amount of Aβ42 comprising a therapeutically effective amount of a mGluR5 NAM, preferably wherein the mGluR5 NAM is selected from Table 1 or Table 2, preferably wherein the mGluR5 NAM is compound 1, 2 or 3 described herein.

Embodiment 16: A composition for use in preserving or preventing intra-ventricular septal thickening in an individual preferably an obese, or pre-diabetic, or diabetic or elderly individual, more preferably an obese individual, or an individual having an elevated amount of Aβ42, more preferably an elevated plasma amount of Aβ42 comprising a therapeutically effective amount of a mGluR5 NAM, wherein the mGluR5 NAM is selected from Table 1 or Table 2, preferably wherein the mGluR5 NAM is compound 1, 2 or 3 described herein.

Embodiment 17: A composition for use in preserving or preventing an increase in left ventricular mass in an individual preferably an obese, or pre-diabetic, or diabetic or elderly individual, more preferably an obese individual, or an individual having an elevated amount of Aβ42, more preferably an elevated plasma amount of Aβ42 comprising a therapeutically effective amount of a mGluR5 NAM, preferably wherein the mGluR5 NAM is selected from Table 1 or Table 2, preferably wherein the mGluR5 NAM is compound 1, 2 or 3 described herein.

Embodiment 18: A composition for use in preventing or treating cardiomyopathy, more preferably diabetic cardiomyopathy, or hypertrophic cardiomyopathy, or ischemic cardiomyopathy, or hypertensive cardiomyopathy in an individual, preferably an obese, or pre-diabetic, or diabetic or elderly individual, more preferably an obese individual, or an individual having an elevated amount of Aβ42, more preferably an elevated plasma amount of Aβ42 comprising a therapeutically effective amount of a mGluR5 NAM, wherein the mGluR5 NAM is selected from Table 1 or Table 2, preferably wherein the mGluR5 NAM is compound 1, 2 or 3 described herein.

Embodiment 19: A composition for use in preventing the reduction of cardiac glucose uptake, or for preventing the accumulation of cardiac tri-acyl glycerol in an individual, preferably an obese, or pre-diabetic, or diabetic or elderly individual, more preferably an obese individual, or an individual having an elevated amount of Aβ42, more preferably an elevated plasma amount of Aβ42 comprising a therapeutically effective amount of a mGluR5 NAM, preferably wherein the mGluR5 NAM is selected from Table 1 or Table 2, preferably wherein the mGluR5 NAM is compound 1, 2 or 3 described herein.

Embodiment 20: A composition for use in preventing or treating obesity-associated cardiomyopathy in an individual, more preferably in an individual having an elevated amount of Aβ42, more preferably an elevated amount of plasma Aβ42 comprising a therapeutically effective amount of a mGluR5 NAM, preferably wherein the mGluR5 NAM is selected from Table 1 or Table 2, preferably wherein the mGluR5 NAM is compound 1, 2 or 3 described herein.

Embodiment 21: A composition for use in preventing a reduction in Peak E wave in an individual, preferably an obese, or pre-diabetic, or diabetic or elderly individual, more preferably an obese individual, or an individual having an elevated amount of Aβ42, more preferably an elevated plasma amount of Aβ42 comprising a therapeutically effective amount of a mGluR5 NAM, preferably wherein the mGluR5 NAM is selected from Table 1 or Table 2, preferably wherein the mGluR5 NAM is compound 1, 2 or 3 described herein.

Embodiment 22: A composition for use in preventing a reduction in cardiac performance in an individual, preferably an obese, or pre-diabetic, or diabetic or elderly individual, more preferably an obese individual, or an individual having an elevated amount of Aβ42, more preferably an elevated plasma amount of Aβ42 comprising a therapeutically effective amount of a mGluR5 NAM, preferably wherein the mGluR5 NAM is selected from Table 1 or Table 2, preferably wherein the mGluR5 NAM is compound 1, 2 or 3 described herein.

### DETAILED DESCRIPTION OF THE INVENTION

The isovolumetric relaxation phase is an essential phase of normal diastole. It is energy dependent, and aberrations of the relaxation phase, as observed in LVDD and related clinical manifestations such as concentric hypertrophy and later heart failure, occur where there is an impairment in availability of ATP, for example as occurring where there is reduced cardiac glucose uptake.

It has been found herein that chronic exposure to Aβ42 induces a cardiac inflammatory response and NLRP3 inflammasome assembly, and that Protein kinase D (PKD) regulates Aβ₄₂-induced inflammasome priming, cardiomyocyte metabolism and cardiac function in obesity.

It has also been found herein that chronic exposure to Aβ42 results in impairments in cardiac metabolism, including a reduction in cardiac glucose uptake, accumulation in cardiac TAG and impairment in cardiac function including concentric hypertrophy. Importantly, these outcomes are minimised by minimising the exposure of cardiomyocytes to Aβ42 particularly in those individuals having a high fat content diet and/or whom are overweight of obese. In particular, as exemplified herein, the administration of mGluR5 NAM prevents a decline in cardiac diastolic function and otherwise prevents Aβ42-mediated decrements in overall cardiac performance.

Without wanting to be bound by hypothesis it is believed that chronic exposure to Aβ42 causes or otherwise results in cardiomyocyte inflammation leading to impaired cardiomyocyte metabolism, reducing their glucose uptake and shunting of glucose into TAG and TAG accumulation, and that minimisation of exposure of cardiomyocytes to Aβ42 reduces these pathological outcomes.

Further, these outcomes are believed to arise from increased or aberrant interaction of Aβ42 with mGluR5 on cardiomyocytes in individuals having chronic exposure to plasma Aβ42. Aβ42 is a mGluR5 ligand (Haas L. T. et al. 2014 JBC 289:28460-28477, mGluR5 agonism increases PKD activity (Krueger D. D. et al. 2010 J. Mol. Neurosci. 42(1):1-8; Fu Y. and Rubin C. S. 2011 EMBO 12:785-796) and PKD phosphorylates NLRP3, leading to NLRP3 inflammasome assembly (Zhang et al. 2017 J Exp Med. 14(9):2671-2693).

Further mGluR5 NAM are utilised herein to minimise the cardiomyocyte mGluR5 response to binding Aβ42, particularly Aβ42 produced by adipocytes, in individuals in whom the prevention or treatment of LVDD is required.

### 1. DEFINITIONS

For purposes of interpreting this specification, the following definitions will apply and whenever appropriate, terms used n the singular will also include the plural and vice versa.

As used herein, the term “about” in relation to a numerical value x means+/−10%, unless the context dictates otherwise.

As used herein, the term “Amyloid beta” (Aβ or Abe) denotes peptides of 3543 amino acids, preferably Aβ42, that are crucially involved in Alzheimer's disease as the main component of the amyloid plaques found in the brains of Alzheimer patients. The peptides derive from the amyloid precursor protein (APP), which is cleaved by beta secretase and gamma secretase to yield Aβ. Aβ molecules can aggregate to form flexible soluble oligomers which may exist in several forms.

As used herein, the term “mGluR5” or “mGlu5” or “metabotropic glutamate receptor 5” generally refers to a receptor of Group I of the metabotropic glutamate receptor family. mGluR5 is generally found post-synaptically within the brain, but is also expressed in other tissues including the heart. mGluR5 binds to L-glutamate, the major excitatory neurotransmitter of the mammalian central nervous system. mGluR5 couples to the heterotrimer G protein, G_{q}/G₁₁, and activate phospholipase C_{β}, resulting in the hydrolysis of phosphoinositides and generation of inositol 1,4,5-trisphosphate (IP₃) and diacyl-glycerol (DAG). This classical pathway leads to calcium mobilization and activation of protein kinase C (PKC). However, it is now recognized that these receptors can modulate additional signaling pathways including other cascades downstream of G_{q} as well as pathways stemming from G_{i/o}, Gₛ, and other molecules independent of G proteins. Depending on the cell type, group I mGluRs can activate a range of downstream effectors, including phospholipase D, protein kinase pathways such as casein kinase 1, cyclin-dependent protein kinase 5, Jun kinase, components of the mitogen-activated protein kinase/extracellular receptor kinase (MAPK/ERK) pathway, and the mammalian target of rapamycin (MTOR)/p70 S6 kinase pathway. The latter pathways, MAPK/ERK and MTOR/p70 S6 kinase, are thought to be particularly important for the regulation of synaptic plasticity by group I mGluRs. DAG biogenesis arising from mGluR5 activation can lead to PKD activation, resulting in phosphorylation of PKD within its catalytic domain at Ser-744 and Ser-748, as well as at the auto-phosphorylation site Ser-916. mGluR5 also serves as a receptor for both cellular prior protein (PrP^{c}) and Aβ42, which activate mGluR5 to release Ca²⁺ from intracellular stores. The structure of mGluR5 comprises a 7 TM α-helical domain that is connected to a large bi-lobed extracellular amino-terminal domain via a cysteine rich region. While the orthosteric binding site is found in the extracellular N-terminal domain, the allosteric binding sites tend to be located in the transmembrane domain.

A “mGluR5 negative allosteric modulator” or “mGluR5 NAM” as used herein generally refers to a compound that binds at a site distinct from the mGluR5 orthosteric site, thereby inhibiting the response of mGluR5 to binding of a native ligand (for example Aβ42) to the receptor.

As used herein, the term “diastolic dysfunction” generally refers to a condition characterised by the inability of the left ventricle to fill an adequate end diastolic volume at a physiologically normal or acceptable pressure.

As used herein, the term “E/A ratio” generally refers to the ratio of the E wave to the A wave. On echocardiography, the peak velocity of blood flow across the mitral valve during early diastolic filling corresponds to the E wave. Similarly, atrial contraction corresponds to the A wave. From these findings, “the E/A ratio” is calculated. Under normal conditions, E is greater than A and the E/A ratio is approximately 1.5. In early diastolic dysfunction, relaxation is impaired and, with vigorous atrial contraction, the E/A ratio decreases to less than 1.0. As the disease progresses, left ventricular compliance is reduced, which increases left atrial pressure and, in turn, increases early left ventricular filling despite impaired relaxation. This paradoxical normalization of the E/A ratio may be called “pseudonormalization”. In patients with severe diastolic dysfunction, left ventricular filling occurs primarily in early diastole, creating an E/A ratio greater than 2.0.

As used herein, “deceleration time” is the time taken from the maximum E point to baseline. In adults, it is normally less than 220 milliseconds.

As used herein, the term “concentric hypertrophy” generally refers to a form of cardiac hypertrophy associated with increased left ventricular wall thickness, or associated with an increase in LV mass without dilation of the LV, for example as measured by LVIDd. An increase in pressure, common in hypertension or resistance training, results in a concentric hypertrophic phenotype. Concentric hypertrophy differs from “eccentric hypertrophy”, the latter being characterised by dilatation of the left ventricular chamber and is observed in, or associated with valvular defects or endurance training. Eccentric hypertrophy may develop from concentric hypertrophy. An individual with diastolic dysfunction, in particular, an individual with early stage diastolic dysfunction may or may not have detectable concentric hypertrophy.

As used herein, the term “HFpEF” or “heart failure with preserved ejection fraction” generally refers to a form of heart failure characterised by normal ejection fraction (at or above about 50% of ventricle volume) dependent on increased LV pressure.

As used herein, “Cardiomyopathy” generally refers to a heterogeneous group of diseases of the myocardium associated with mechanical and/or electrical dysfunction, which usually (but not invariably) exhibit inappropriate ventricular hypertrophy or dilatation. Cardiomyopathy may be a primary cardiomyopathy, which is confined to the heart, preferably an acquired cardiomyopathy, more preferably an obesity-associated cardiomyopathy. An obesity-associated cardiomyopathy is defined myocardial disease in obese individuals that cannot be explained by diabetes mellitus, hypertension, coronary artery disease or other etiologies. The presentation of this disease can vary from asymptomatic left ventricular dysfunction to overt dilated cardiomyopathy and heart failure.

As used herein, the term “elderly individual” refers to an individual over 60 years of age, more preferably 65 or 70 or 75 years of age.

As used herein, the term “pharmaceutically acceptable” means a nontoxic material that does not interfere with the effectiveness of the biological activity of the active ingredient(s).

As used herein, the term “treat”, “treating” or “treatment” in connection to a disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, {e.g., stabilization of a discernible symptom), physiologically, {e.g., stabilization of a physical parameter), or both. The term “alleviating” or “alleviation”, for example in reference to a symptom of a condition, as used herein, refers to reducing at least one of the frequency and amplitude of a symptom of a condition in a patient. In one embodiment, the terms “method for the treatment” or “method for treating”, as used herein, refer to “method to treat”.

As used herein, the term “therapeutically effective amount” refers to an amount of the compound of the invention, e.g. mGluR5 NAM which is sufficient to achieve the stated effect. Accordingly, a therapeutically effective amount of a mGluR5 NAM; will be an amount sufficient for the treatment or prevention of the condition mediated by or associated with Aβ42 plasma expression or production.

By “therapeutic regimen” is meant the pattern of treatment of an illness, e.g., the pattern of dosing used during the treatment of the disease or disorder.

As used herein, a subject is “in need of” a treatment if such subject would benefit biologically, medical or in quality of life from such treatment.

### 2. DETAILED DESCRIPTION OF THE FIGURES

FIG. 1—Chronic Aβ42 administration alters cardiac metabolism.

FIG. 2—Chronic Aβ42 administration alters cardiac function.

FIG. 3—Administration of anti-Aβ42 antibodies preserves diastolic function in development of obesity.

FIG. 4—Administration of anti-Aβ42 antibodies prevents concentric hypertrophy in development of obesity.

FIG. 5—Aβ₄₂ induces a cardiac inflammatory response and NLRP3 inflammasome assembly.

FIG. 6—Protein kinase D inhibition prevents Aβ₄₂-induced inflammasome priming.

FIG. 7—PKD regulates cardiomyocyte metabolism.

FIG. 8—Reduced PKD activity preserves cardiac function in obesity.

FIG. 9—mGluR5 NAM prevents development of diastolic dysfunction and prevents a decline in myocardial performance associated with chronic administration of Aβ42.

FIG. 10—Administration of anti-Aβ42 antibodies preserves diastolic function in established obesity.

FIG. 11—Chronic 440 administration does not alter cardiac function.

FIG. 12—Administration of mGluR5 NAM preserves diastolic function in established obesity.

### 3. MODES OF CARRYING OUT THE INVENTION

### 3.1 Individuals

An individual to whom the methods of the invention are applied is mammalian, preferably a human being.

An individual may not have diastolic dysfunction at the time of treatment. Such an individual may be at risk for diastolic dysfunction i.e. may have one or more risk factors for diastolic dysfunction. For example, the individual may be pre diabetic or diabetic, overweight or obese, female, have Alzheimer's disease or other neural disease with Aβ involvement, or elderly. The individual may have an elevated amount of Aβ42, preferably an elevated amount of plasma Aβ42. The invention may be applied to such an individual to prevent the development of diastolic dysfunction, or to prevent diastolic dysfunction.

In another embodiment, an individual may have diastolic dysfunction at the time of treatment. Such an individual may be asymptomatic for diastolic dysfunction, or symptomatic for diastolic dysfunction. The invention may be applied to such an individual to treat or ameliorate or alleviate diastolic dysfunction.

In one embodiment, the individual to be administered an mGluR5 NAM is obese and has an elevated amount of plasma Aβ42 and may or may not have diastolic dysfunction. Such an individual may have obesity associated cardiomyopathy, or may be at risk for same.

Stages of diastolic dysfunction have been classified according to various grading systems. For example, four basic echocardiographic patterns of diastolic dysfunction, (graded I to IV) according to the American Society of Echocardiography and the European Association of Cardiovascular Imaging are described:
Grade I diastolic dysfunction. On the mitral inflow Doppler echocardiogram, the E/A ratio is ≤0.8 and deceleration time is >200 ms, while the E/e′ ratio, a measure of the filling pressure, is within normal limits at <10. This pattern may develop normally with age in some patients, and many grade I patients will not have any clinical signs or symptoms of heart failure.
Grade II diastolic dysfunction is called “pseudonormal filling dynamics” with the E/A ratio between 0.8 and 2.0, and a reduction in deceleration time to between 160 and 220 ms. This is considered moderate diastolic dysfunction and is associated with elevated left atrial filling pressures, with an E/e′ ration between 10 and 14. These patients more commonly have symptoms of heart failure, and many have left atrial enlargement due to the elevated pressures in the left heart.
Class III diastolic dysfunction patients have an E/A ratio >2 and E/e′ ratio >14. They will demonstrate reversal of their diastolic abnormalities on echocardiogram when they perform the Valsalva maneuver. This is referred to as “reversible restrictive diastolic dysfunction”.
Class IV diastolic dysfunction patients will not demonstrate reversibility of their echocardiogram abnormalities, and are therefore said to suffer from “fixed restrictive diastolic dysfunction”.

Grade III and IV diastolic dysfunction are called “restrictive filling dynamics”. These are both severe forms of diastolic dysfunction, and patients tend to have advanced heart failure symptoms.

In one embodiment, an individual having Grade I diastolic dysfunction (as described above), preferably having an elevated plasma amount of Aβ42 is provided with a mGluR5 NAM to prevent the development of more severe diastolic dysfunction, or otherwise to preserve diastolic function.

In one embodiment, an individual having Grade II, III or IV diastolic dysfunction (as described above), preferably having an elevated plasma amount of Aβ42 is provided with a mGluR5 NAM to treat or reverse diastolic dysfunction, or to treat or reverse one or more symptoms or characters of diastolic dysfunction.

In one embodiment, an individual may have concentric hypertrophy.

An individual in need of treatment may have a normal left ventricle diameter and may have a normal cardiac weight.

An individual in need of treatment may have an increased LV deceleration time.

An individual in need of treatment may have a decreased E wave i.e. reduced peak velocity of blood flow across the mitral valve during early diastolic filling.

An individual in need of treatment may have a cardiomyopathy, especially an ischemic or hypertrophic cardiomyopathy.

An individual in need of treatment may have a systolic condition in addition to diastolic dysfunction.

An individual the subject of treatment may be symptomatic for heart failure and may be symptomatic for HFPpEF or may be asymptomatic for heart failure or HFpEF. Symptoms of heart failure generally include shortness of breath including exercise induced dyspnea, paroxysmal nocturnal dyspnea and orthopnea, exercise intolerance, fatigue, elevated jugular venous pressure, and edema. Patients with HFpEF poorly tolerate stress, particularly hemodynamic alterations of ventricular loading or increased diastolic pressures. Often there is a more dramatic elevation in systolic blood pressure in HFpEF.

An individual who is asymptomatic or symptomatic for heart failure may or may not be obese or overweight, diabetic or pre-diabetic, have Alzheimer's disease or other neural disease with Aβ involvement, or elderly.

### 3.2 Screening Individuals for LVDD

In a particularly preferred embodiment, an individual may be selected for treatment or prevention of LVDD, or screened for LVDD, or assessed for risk of developing LVDD by assessing or measuring the plasma amount of Aβ and optionally comparing with a normal control describing an amount of Aβ in plasma in an individual not having, or not at risk of having diastolic dysfunction, for example, an individual who is not overweight or obese, or not pre-diabetic or diabetic, or who does not have Alzheimer's disease or who is not elderly.

In one embodiment, a control may be an age matched control. Where the individual to be assessed is elderly, the control may describe an amount of Aβ42 in plasma that is consistent with that found in a normal individual having an age of about 20 to 40 years old.

In one embodiment, a control describes the amount of Aβ42 in plasma from an individual having a body mass index in the normal range, from about 18.5 to 24.9 kg/m².

In one embodiment, a control describing the amount of Aβ42 in plasma may be derived from a single individual. In another embodiment, a control may be derived from a cohort of individuals.

It has been established in the Examples herein that diastolic dysfunction is induced by administration of an amount of about 0.04 mg/kg of Aβ42 peptide per day. Further, individuals on a high fat diet may develop a plasma amount of Aβ42 peptide of about 3 fold above controls. In one embodiment, an individual to be selected for treatment may have a plasma amount of Aβ42 peptide of about 10 to 100 pM, or about 1 to at least 10 fold the amount of Aβ42 peptide in a control.

A control may provide a reference point against which a determination regarding implementation of subsequent prophylaxis or therapy can be made. The determination may be made on the basis of the comparison between test sample obtained from the individual being assessed for prophylaxis or treatment and the control.

In certain embodiments, the control may be provided in the form of data that has been derived by another party, and/or prior to assessment of the subject for treatment. For example, the control may be derived from a commercial database or a publically available database.

In one embodiment the individual is selected for treatment or prevention of LVDD, or screened for LVDD, or assessed for risk of developing LVDD, where the individual has an amount of Aβ or fragment thereof, preferably Aβ42 that is greater than the amount of Aβ or fragment thereof, preferably Aβ42 in a normal control.

Methods for measurement of plasma amounts of Aβ or fragment thereof such as Aβ42 are known in the art: [Kim et al., Sci. Adv. 2019; 5:eaav1388 17 Apr. 2019; Shie, F S et al., PLOSONE|DOI:10.1371/journal.pone.0134531 Aug. 5, 2015; Balakrishnan K et al. Journal of Alzheimer's Disease 8 (2005) 269-282; Luciano R et al., PEDIATRICS Volume 135, number 6, June 2015]

In certain embodiments, the samples to be tested are body fluids such as blood, serum, plasma, urine, tears, saliva, CSF and the like.

In certain embodiments, the sample from the individual may require processing prior to detection of the levels of Aβ42. For example, the sample may be centrifuged or diluted to a particular concentration or adjusted to a particular pH prior to testing. Conversely, it may be desirable to concentrate a sample that is too dilute, prior to testing.

In certain embodiments Aβ42 may be measured, or peptides or complexes that comprise Aβ42 may be measured.

In other embodiments, fragments of Aβ42 comprising the Aβ42 C-terminal sequences that distinguish Aβ42 from Aβ40 may be measured.

The above described methods may be combined with the following diagnostic procedures for detecting, assessing or measuring diastolic dysfunction or related heart failure such as HFPeF, or the following procedures may be used without assessment of plasma amount of Aβ42.

Two-dimensional echocardiography with Doppler flow measurements is commonly used to assess diastolic dysfunction. Exercise may be required to clearly demonstrate diastolic functional changes. During diastole, blood flows through the mitral valve when the LV relaxes, causing an early diastolic mitral velocity (E), and then additional blood is pumped through the valve when the left atrium contracts during late diastole (A). The E/A ratio can be altered in diastolic dysfunction.

Tissue Doppler imaging is an echocardiographic technique that measures the velocity of the mitral annulus. This velocity has been shown to be a sensitive marker of early myocardial dysfunction. With abnormal active relaxation, mitral annulus velocity during early diastole (E) is decreased while mitral annulus velocity during late diastole (A) is increased, resulting in a lowered E/A ratio. In animal models, tissue Doppler imaging has been validated as a reliable tool for the evaluation of diastolic dysfunction.

The E- and A-wave velocities are affected by blood volume, mitral valve anatomy, mitral valve function, and atrial fibrillation, making standard echocardiography less reliable. In these cases, tissue Doppler imaging is useful for measuring mitral annular motion (a measure of transmitral flow that is independent of the aforementioned factors). Cardiac catheterization remains the preferred method for diagnosing diastolic dysfunction. However, in day-to-day clinical practice, two-dimensional echocardiography with Doppler is the best noninvasive tool to confirm the diagnosis. Rarely, radionuclide angiography is used for patients in whom echocardiography is technically difficult.

LV inflow propagation velocity (VP) by color M-mode Doppler is another relatively preload-insensitive index of LV relaxation. It has been shown to correlate well with the time constant of isovolumic relaxation (τ), both in animals and humans.

Recently, speckle tracking echocardiography (STE) has emerged as a promising technique for the evaluation of myocardial wall motion by strain analysis. By tracking the displacement of speckles during the cardiac cycle, STE allows semiautomated delineation of myocardial deformation.

Cardiac magnetic resonance (CMR) imaging is a newer technique for measuring diastolic dysfunction. Myocardial tagging allows the labeling of specific myocardial regions. Following these regions during diastole enables them to be analyzed in a manner similar to STE. In addition, the rapid diastolic untwisting motion followed by CMR tagging is directly related to isovolumic relaxation and can be used as an index of the rate and completeness of relaxation.

Biomarkers may also be assessed for diagnosis of LVDD. B-type natriuretic peptide (BNP) and TnI have been used as HF biomarkers and exhibit strong association with hospitalization.

Nevertheless, they are nonspecific and not well correlated with diastolic dysfunction. Recently, it has been reported that cMyBP-C could be a new biomarker releases from damaged myofilaments. Additionally, elevated S-glutathionylated cMyBP-C level can be detected in the blood of patients with diastolic dysfunction. Hypertension and diabetes lead to cardiac oxidation and S-glutathionylation of cMyBP-C, a cardiac contractile protein, which leads to impaired relaxation, and modified cMyBP-C in the blood may represent a circulating biomarker for diastolic dysfunction.

3.3 mGluR5 Negative Allosteric Modulators

mGluR5 NAM for use in the invention generally minimise the response of mGluR5 binding to a ligand such as Aβ42 or Aβ/Prp^{c}.

Without wanting to be bound by hypothesis, it is believed that the chronic plasma Aβ42 exposure leads to aberrant or abnormal exposure of cardiomyocyte mGluR5 molecules to Aβ42, leading to a reprogramming of these cells, reducing their glucose uptake and shunting of glucose into TAG, and that the administration of a mGluR5 NAM minimises the mGluR5 response to binding Aβ42, resulting in a minimisation of diastolic dysfunction, preferably through a minimisation of Aβ induced or associated cardiomyocyte inflammation and/or reduced cardiac glucose uptake.

A mGluR5 NAM for use in the invention may minimise the activation of Protein Kinase D (PKD) that otherwise occurs where mGluR5 has bound to Aβ42 in the absence of a mGluR5 NAM.

A mGluR5 NAM for use in the invention may minimise the assembly or production of the NPRL3 inflammasome that otherwise occurs where mGluR5 has bound to Aβ42 in the absence of a mGluR5 NAM.

A mGluR5 NAM for use in the invention may minimise the cardiomyocyte gene expression of any one or more of the following genes: IL-6, IL-1p, TNF-α, MCP-1, CCI2, NLRP3, ASC, IFNα, Casp 1 and IL-18 in the presence of elevated plasma Aβ42.

A mGluR5 NAM for use in the invention may minimise the cardiomyocyte expression or production of pro or mature Caspase 1 protein.

A mGluR5 NAM for use in the invention may improve or prevent impaired cardiac glucose uptake or metabolism, such impairment that otherwise occurs where mGluR5 has bound to Aβ42 in the absence of a mGluR5 NAM.

A mGluR5 NAM for use in the invention may improve or prevent impaired cardiac function, such impairment that otherwise occurs where mGluR5 has bound to Aβ42 in the absence of a mGluR5 NAM.

There now follows a discussion of mGluR5 NAM contemplated for use in the invention.

### 3.3.1 Alkyne-Containing Compounds

Examples of alkyne containing compounds contemplated for use as mGluR5 NAM according to the invention are described in Table 1 and in the patent specifications referred to therein. The entire contents of the patent specifications referred to in Table 1 are incorporated herein by reference.

TABLE 1

| Compound # | Structure | Reference |
|---|---|---|
| 1 | 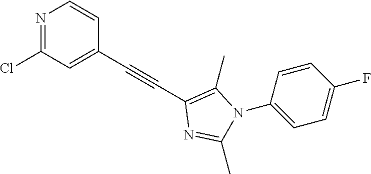 [SMILES: Cc1nc(C#Cc2ccnc(Cl)c2)c(C)n1-c1ccc(F)cc1] | Basimglurant CAS # 802906-73-6 |
| 2 | 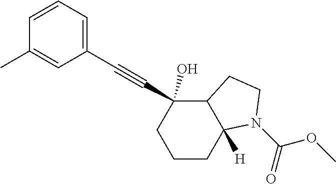 | Mavoglurant CAS # 543906-09-8 |
| 3 | 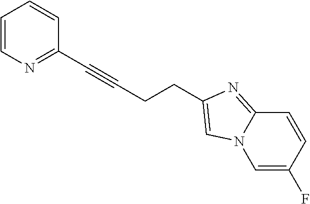 [SMILES: Fc1ccc2nc(CCC#Cc3ccccn3)cn2c1] | Dipraglurant CAS # 872363-17-2 |
| 4 | 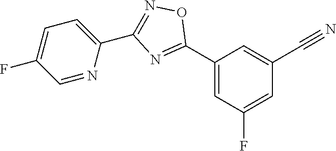 [SMILES: N#Cc1cc(F)cc(-c2nc(-c3ccc(F)cn3)no2)c1] | AZD9272 CAS # 327056-26-8 |
| 5 | 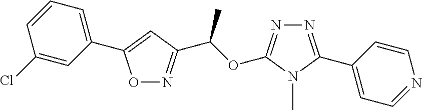 [SMILES: C[C@@H](Oc1nnc(-c2ccncc2)n1C)c1cc(-c2cccc(Cl)c2)on1] | AZD2066 CAS # 934282-55-0 |
| 6, 7 | 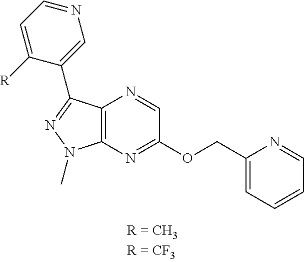 | PF-06297470 CAS # 1539296-45-1 PF-06422913 CAS # 1539296-46-2 |
| 8 | 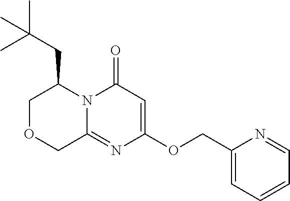 [SMILES: CC(C)(C)C[C@@H]1COCc2nc(OCc3ccccn3)cc(=O)n21] | PF-06462894 CAS # 1622291-66-0 |
| 21 | 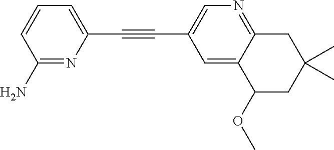 [SMILES: COC1CC(C)(C)Cc2ncc(C#Cc3cccc(N)n3)cc21] | EP2650284 |
| 22 | 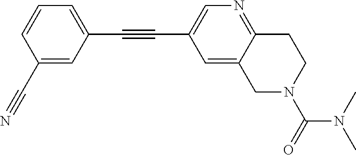 [SMILES: CN(C)C(=O)N1CCc2ncc(C#Cc3cccc(C#N)c3)cc2C1] | EP2650284 |
| 23 | 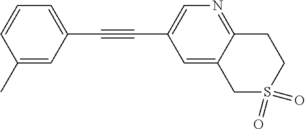 [SMILES: Cc1cccc(C#Cc2cnc3c(c2)CS(=O)(=O)CC3)c1] | EP2650284 |
| 24 | 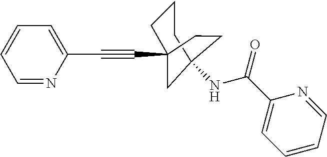 [SMILES: O=C(N[C@]12CCC[C@](C#Cc3ccccn3)(CC1)C2)c1ccccn1] | WO2013/040535 |
| 25 | 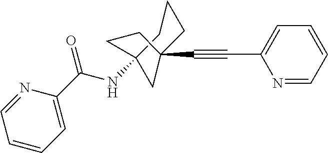 [SMILES: O=C(N[C@@]12CCC[C@@](C#Cc3ccccn3)(CC1)C2)c1ccccn1] | WO2013/040535 |
| 26 | 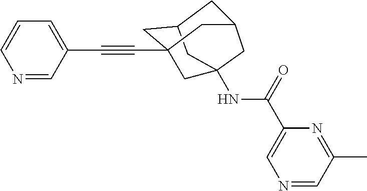 [SMILES: Cc1cncc(C(=O)NC23CC4CC(CC(C#Cc5cccnc5)(C4)C2)C3)n1] | WO2013/040535 |
| 27 & 28 | 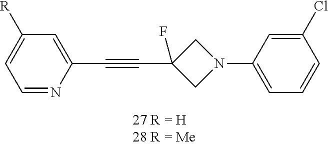 | WO2014/124560 |
| 29, 30, 31 | 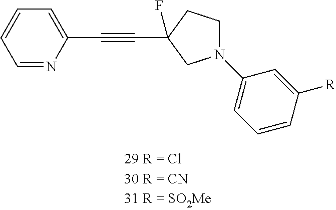 | WO2014/124560 |
| 32, 33 | 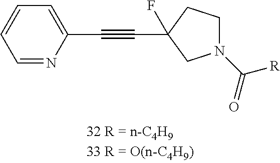 | WO2014/124560 |
| 34, 35 | 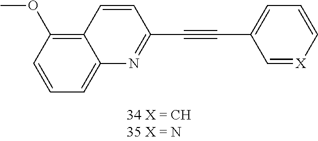 | US2014/0206876 |
| 36, 37 | 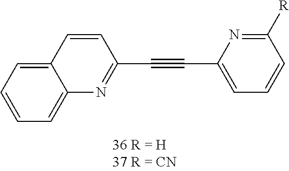 | US2014/0206876 |

### 3.3.2 Non Alkyne Containing Compounds

Examples of non alkyne containing compounds contemplated for use as mGluR5 NAM according to the invention are described in Table 2 and in the patent specifications referred to therein. The entire contents of the patent specifications referred to in Table 2 are incorporated herein by reference.

TABLE 2

| Compound # | Structure | Reference |
|---|---|---|
| 63 | 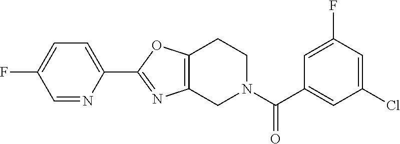 [SMILES: O=C(c1cc(F)cc(Cl)c1)N1CCc2oc(-c3ccc(F)cn3)nc2C1] | WO2014/065270 |
| 64 | 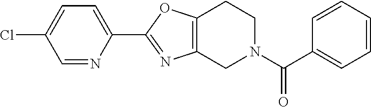 [SMILES: O=C(c1ccccc1)N1CCc2oc(-c3ccc(Cl)cn3)nc2C1] | WO2014/065270 |
| 65 | 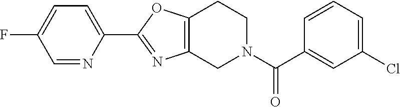 [SMILES: O=C(c1cccc(Cl)c1)N1CCc2oc(-c3ccc(F)cn3)nc2C1] | WO2014/065270 |
| 66 | 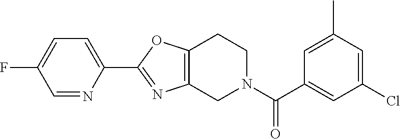 [SMILES: Cc1cc(Cl)cc(C(=O)N2CCc3oc(-c4ccc(F)cn4)nc3C2)c1] | WO2014/065270 |
| 67, 68 | 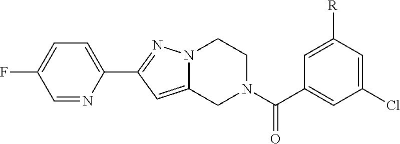 | JP 2015059118 |
| 69 | 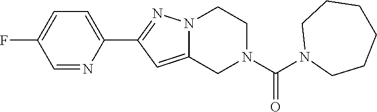 [SMILES: O=C(N1CCCCCC1)N1CCn2nc(-c3ccc(F)cn3)cc2C1] | JP 2015059118 |
| 70 | 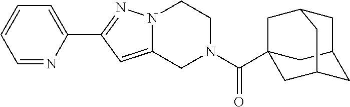 [SMILES: O=C(N1CCn2nc(-c3ccccn3)cc2C1)C12CC3CC(CC(C3)C1)C2] | JP 2015059118 |
| 71 | 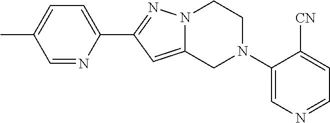 [SMILES: Cc1ccc(-c2cc3n(n2)CCN(c2cnccc2C#N)C3)nc1] | JP 2015059118 |
| 72 | 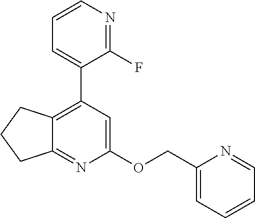 [SMILES: Fc1ncccc1-c1cc(OCc2ccccn2)nc2c1CCC2] | WO2014/034898 |
| 73 | 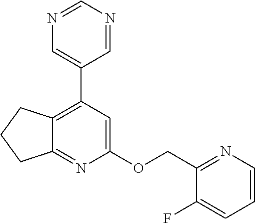 [SMILES: Fc1cccnc1COc1cc(-c2cncnc2)c2c(n1)CCC2] | WO2014/034898 |
| 74 | 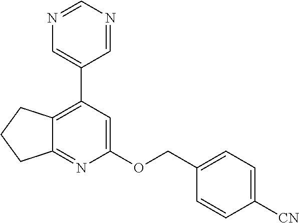 [SMILES: N#Cc1ccc(COc2cc(-c3cncnc3)c3c(n2)CCC3)cc1] | WO2014/034898 |
| 75 | 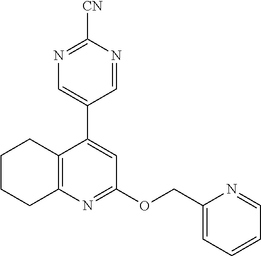 [SMILES: N#Cc1ncc(-c2cc(OCc3ccccn3)nc3c2CCCC3)cn1] | WO2014/034898 |
| 76 | 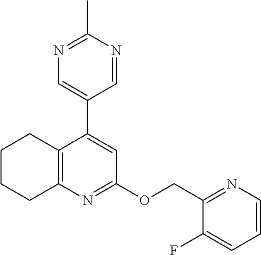 [SMILES: Cc1ncc(-c2cc(OCc3ncccc3F)nc3c2CCCC3)cn1] | WO2014/034898 |
| 77 | 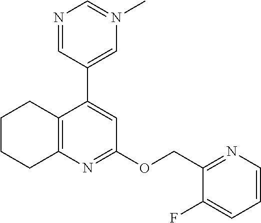 | WO2014/034898 |
| 78 | 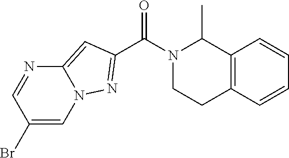 [SMILES: CC1c2ccccc2CCN1C(=O)c1cc2ncc(Br)cn2n1] | WO2012/139876 WO2008/015269 |
| 79 | 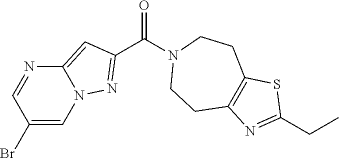 [SMILES: CCc1nc2c(s1)CCN(C(=O)c1cc3ncc(Br)cn3n1)CC2] | WO2013/144172 |
| 80 | 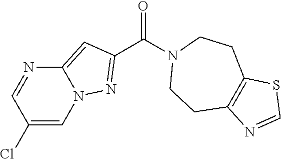 [SMILES: O=C(c1cc2ncc(Cl)cn2n1)N1CCc2ncsc2CC1] | WO2013/144172 |
| 81 | 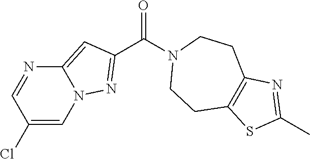 [SMILES: Cc1nc2c(s1)CCN(C(=O)c1cc3ncc(Cl)cn3n1)CC2] | WO2013/144172 |
| 82 | 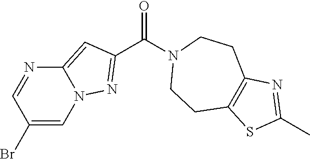 [SMILES: Cc1nc2c(s1)CCN(C(=O)c1cc3ncc(Br)cn3n1)CC2] | WO2013/144172 |
| 83 | 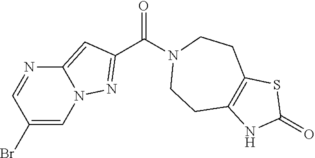 [SMILES: O=C(c1cc2ncc(Br)cn2n1)N1CCc2[nH]c(=O)sc2CC1] | WO2013/144172 |
| 84 | 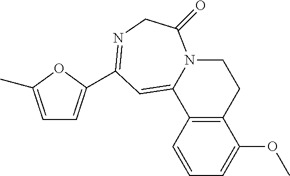 | WO2014/030128 |
| 85 | 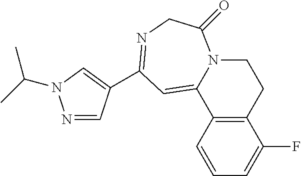 [SMILES: CC(C)n1cc(C2=NCC(=O)N3CCc4c(F)cccc4C3=C2)cn1] | WO2014/030128 |
| 86 | 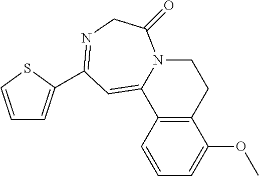 [SMILES: COc1cccc2c1CCN1C(=O)CN=C(c3cccs3)C=C21] | WO2014/030128 |
| 87 | 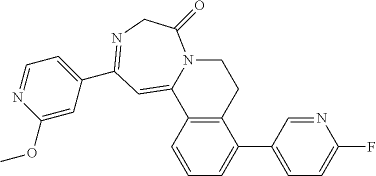 [SMILES: COc1cc(C2=NCC(=O)N3CCc4c(cccc4-c4ccc(F)nc4)C3=C2)ccn1] | WO2014/030128 |
| 88 | 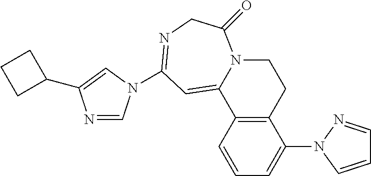 [SMILES: O=C1CN=C(n2cnc(C3CCC3)c2)C=C2c3cccc(-n4cccn4)c3CCN12] | WO2014/030128 |
| 89 | 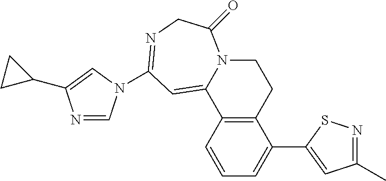 [SMILES: Cc1cc(-c2cccc3c2CCN2C(=O)CN=C(n4cnc(C5CC5)c4)C=C32)sn1] | WO2014/030128 |
| 90 | 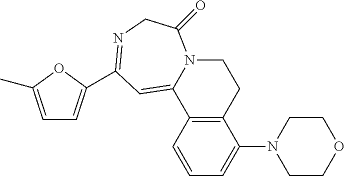 [SMILES: Cc1ccc(C2=NCC(=O)N3CCc4c(cccc4N4CCOCC4)C3=C2)o1] | WO2014/030128 |
| 91 | 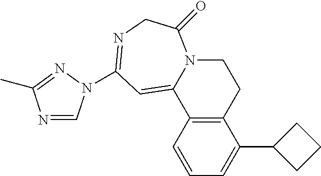 [SMILES: Cc1ncn(C2=NCC(=O)N3CCc4c(cccc4C4CCC4)C3=C2)n1] | WO2014/030128 |
| 92 | 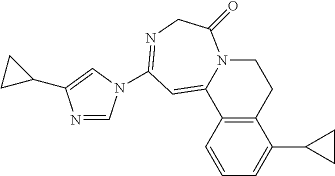 [SMILES: O=C1CN=C(n2cnc(C3CC3)c2)C=C2c3cccc(C4CC4)c3CCN12] | WO2014/030128 |
| 101 | 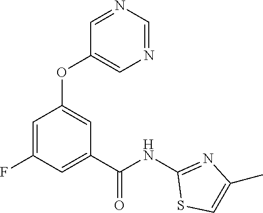 [SMILES: Cc1csc(NC(=O)c2cc(F)cc(Oc3cncnc3)c2)n1] | VU0409106 CAS # 1276617-62-9 |
| 102 | 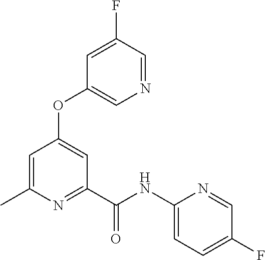 [SMILES: Cc1cc(Oc2cncc(F)c2)cc(C(=O)Nc2ccc(F)cn2)n1] | WO2012/118563 |
| 103 | 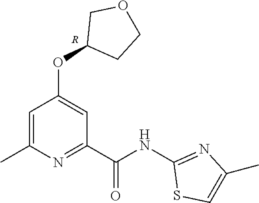 | WO2015/200682 |
| 104 | 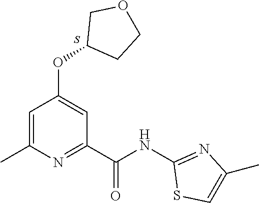 | WO2015/200682 |
| 105 | 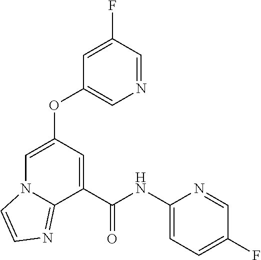 [SMILES: O=C(Nc1ccc(F)cn1)c1cc(Oc2cncc(F)c2)cn2ccnc12] | WO2015/077246 |
| 106 | 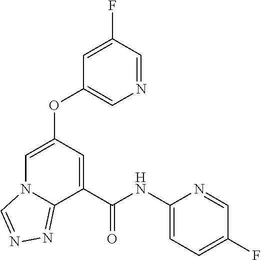 [SMILES: O=C(Nc1ccc(F)cn1)c1cc(Oc2cncc(F)c2)cn2cnnc12] | WO2015/077246 |
| 107 | 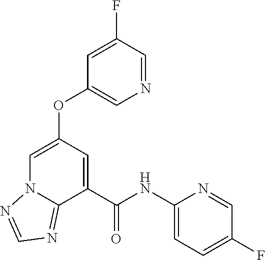 [SMILES: O=C(Nc1ccc(F)cn1)c1cc(Oc2cncc(F)c2)cn2ncnc12] | WO2015/077246 |
| 108 | 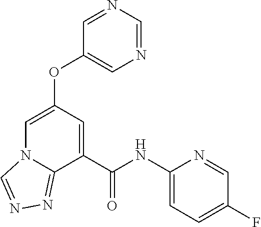 [SMILES: O=C(Nc1ccc(F)cn1)c1cc(Oc2cncnc2)cn2cnnc12] | WO2015/077246 |
| 109 | 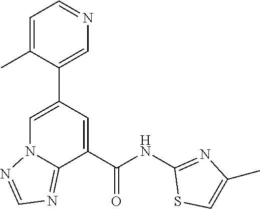 | US20160096833 |
| 110 | 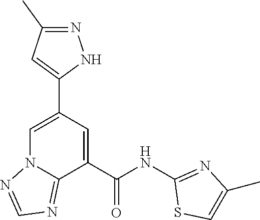 [SMILES: Cc1cc(-c2cc(C(=O)Nc3nc(C)cs3)c3ncnn3c2)[nH]n1] | US20160096833 |
| 111 | 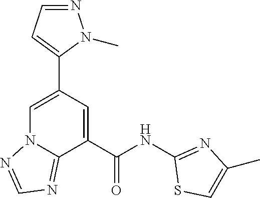 [SMILES: Cc1csc(NC(=O)c2cc(-c3ccnn3C)cn3ncnc23)n1] | US20160096833 |

### 3.4 Pharmaceutical Compositions and Administration

The mGluR5 NAM described herein and the pharmaceutically acceptable salts can be used as therapeutically active substances, e.g. in the form of pharmaceutical preparations. The pharmaceutical preparations can be administered orally, e.g. in the form of tablets, coated tablets, dragees, hard and soft gelatin capsules, solutions, emulsions or suspensions. The administration can, however, also be effected rectally, e.g. in the form of suppositories, or parenterally, e.g. in the form of injection solutions.

The mGluR5 NAM described herein and the pharmaceutically acceptable salts thereof can be processed with pharmaceutically inert, inorganic or organic carriers for the production of pharmaceutical preparations. Lactose, corn starch or derivatives thereof, talc, stearic acids or its salts and the like can be used, for example, as such carriers for tablets, coated tablets, dragees and hard gelatin capsules. Suitable carriers for soft gelatin capsules are, for example, vegetable oils, waxes, fats, semi-solid and liquid polyols and the like. Depending on the nature of the active substance no carriers are however usually required in the case of soft gelatin capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical preparations can, moreover, contain pharmaceutically acceptable auxiliary substances such as preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Medicaments containing a mGluR5 NAM described herein and the pharmaceutically acceptable salts and a therapeutically inert carrier are also provided by the present invention, as is a process for their production, which comprises mGluR5 NAM described herein and the pharmaceutically acceptable salts and, if desired, one or more other therapeutically valuable substances into a galenical administration form together with one or more therapeutically inert carriers.

The dosage can vary within wide limits and will, of course, have to be adjusted to the individual requirements in each particular case. In the case of oral administration the dosage for adults can vary from about 0.01 mg to about 1000 mg per day of a mGluR5 NAM described herein and the pharmaceutically acceptable salts.

The daily dosage may be administered as single dose or in divided doses and, in addition, the upper limit can also be exceeded when this is found to be indicated.

The pharmaceutical preparations may conveniently contain about 1-500 mg, particularly 1-100 mg, of a mGluR5 NAM described herein.

### EXAMPLES

### Example 1—Materials & Methods

Aβ42 administration study: Lyophilised recombinant Aβ₄₂ (Millipore) and scrambled control peptide (ScrAβ₄₂; Millipore) were resuspended in 1% NH₄OH and aliquoted at 200 ng/ml in H₂O and stored at −80° C. for no longer than 4 weeks. Male C57BL6 mice were obtained from the Animal Resource Centre (Perth, Wash.) at 4 weeks of age and housed with 4 mice per cage on a 12 hr light/dark cycle at a temperature of 22° C. and a constant humidity with a normal rodent diet. At 12 weeks of age, mice were grouped according to body mass and composition, determined by EchoMRI. Mice were then administered 1 pg of recombinant Aβ₄₂ or ScrAβ₄₂ (n=10/group per cohort) by i.p. injection once/day for 5 wks. An i.p. glucose tolerance test (GTT) was performed on the final treatment day following an overnight fast. Mice were administered 2 g/kg lean mass of glucose including radioactive glucose tracers, prepared as follows. 100 μl of 1 μCi/μ1 glucose analogue, [³H]-2-deoxyglucose (2-DOG), and 500 μl of 200 μCi/mL U-¹⁴C glucose were evaporated to dryness before redissolving the radioactive tracers in 1 mL of 50% glucose. This produced a 50% glucose solution containing 100 μCi/mL [³H]-2-DOG and 100 μCi/mL U-¹⁴C glucose. The tail tip of each mouse was cut off and the blood glucose concentration of a blood sample was measured using an AccuCheck II glucometer (Roche). The GTT was initiated via intraperitoneal injection of the radiolabelled glucose solution (2 g/kg body weight, 10 uCi/animal) into the overnight-fasted mice. Further blood samples were taken at 15, 30, 45, 60 and 90 minutes after the injection for the measurement of blood glucose. Blood samples (30 μl) were also taken from the tail tip at each time point and diluted in 100 μl of saline. These samples were then centrifuged and the supernatant collected. 50 μl of the supernatant was diluted in 500 μl of distilled water and then suspended in 4 mL of Ultima Gold XR scintillation fluid (Packard Bioscience). Blood radioactivity was determined at each time point by performing liquid scintillation counting on each solution using the Beckman scintillation counter (LS6000 SC). At the conclusion of the GTT, mice were killed via cervical dislocation. Blood was obtained immediately following by cardiac puncture and the heart, and other tissues were immediately removed. Hearts were washed in ice cold PBS and weighed prior to being snap frozen in liquid nitrogen. The heart (30 mg), epididymal fat pad (30 mg and quadriceps skeletal muscle (30 mg) were homogenised in 1.5 ml of distilled water. The homogenate was centrifuged at 3000 rpm for 10 min at 4° C. 400 μl of the supernatant was diluted into 1.6 mL of distilled water and then suspended in 14 mL of Ultima Gold XR scintillation fluid (Packard Bioscience). The radioactivity of each sample (from both [³H]-2-DOG6P and [³H]-2-DOG) was determined by liquid scintillation counting using the Beckman scintillation counter (LS6000 SC).

The ³H radioactivity was used to measure glucose uptake into each tissue.

To determine the incorporation of U-¹⁴C glucose into triglyceride and the total triglyceride content in the heart, an extraction of triglyceride was carried out using a chloroform/methanol mixture. Samples of heart (30 mg) were hand-homogenised in 2 mL of chloroform/methanol (2:1) and the homogeniser rinsed in a further 2 mL of chloroform/methanol (2:1), and the washings being added to the original extract in 10 mL tubes. The tubes were tightly capped and mixed on a rotator overnight to maximise extraction of the triglycerides. 2 ml of 0.6% saline was then added, to facilitate the separation of the organic and aqueous phases, after which the tubes were mixed thoroughly and then centrifuged at 2000 rpm for 10 minutes. The lower chloroform phase (containing triglycerides) was collected and evaporated to dryness under nitrogen at 45° C. The dried extract was then re-dissolved in 250 μl of 100% ethanol, to redissolve the lipid and enable aliquots to dispensed for assay. The amount of U-¹⁴C glucose clearance into the lipid fraction was measured by suspending 100 μl of the triglyceride solution in 5 mL of Ultima Gold XR scintillation fluid (Packard Bioscience), followed by scintillation counting using the Beckman scintillation counter (LS6000 SC). Total triglyceride content was measured using an enzymatic fluorometric assay (BioVision) as per manufacturers' instructions. Lipoprotein lipase was used in an enzymatic reaction to yield fatty acid and glycerol. Quantified glycerol was used as an indirect measure of triglyceride and was normalised to tissue weight.

Total mRNA from the tissues was extracted by homogenizing ˜20-30 milligrams of tissue in 1 ml of Trizol followed by incubation at room temperature (RT) for 5 min. 200 μL of chloroform was added to the homogenate, shaken for 15 seconds and incubated for 1 min at RT before centrifuging at 12,000 g for 10 min at 4° C. for extracting the upper aqueous phase. An equal volume (350 μl for cell lysate/450 μL for tissue) of 70% ethanol was added to cell/tissue samples and they were further purified with RNeasy spin columns (the RNeasy®min i Kit, Qiagen). Complementary DNA (cDNA) was synthesised using the SuperScript™ III transcription system (Invitrogen). cDNA was quantified by OliGreen assay (Quant-iT™ OliGreen® ssDNA Assay Kit; Invitrogen). All primers were designed in-house using the Beacon Primer Designer program software and synthesised by Gene Works (Adelaide, Australia). Primer sequence efficiency was tested over a wide concentration range. Gene expression levels were quantified using the FastStart Universal SYBR Green Master (ROX; Roche Applied-Science) on the MX3005P™ Multiplex Quantitative PCR (QPCR) system (Stratagene). Log-transformed CT values were normalised to cDNA concentration to determine relative gene expression levels.

The effect of Aβ₄₂ administration on cardiac function was assessed in another cohort of 12-week-old, male C57BL6 mice, which were administered Aβ₄₂ or ScrAβ₄₂ (n=10/group per cohort) by i.p. injection once/day for 5 wks. After 4 weeks of peptide administration, cardiac function was assessed by echocardiography as follows. Mice were anaesthetised with inhalation of 1.5% isoflurane anaesthesia and echocardiography was performed using the Phillips HD15 diagnostic ultrasound system with a 15 MHz linear-array transducer by an experienced veterinarian. The velocity of blood flow through the mitral valve was analysed using Doppler mode imaging. These results were used to calculate the deceleration time and E:A ratio. Doppler imaging was also utilised to measure the velocity of blood flow through the aortic valve. The measurements were then used to calculate the ejection time, peak aortic flow and heart rate. M-mode imaging of the left ventricle was used to measure the thickness of the inter-ventricular septum (IVS), left ventricular internal diameter (LVID) and left ventricular posterior wall (LVPW) in both diastole (d) and end-systole (s) as well as systolic measures such as ejection fraction and fractional shortening. An estimation of LV mass was calculated from the m-mode imaging by using the formula (1.05[LVIDd+LVPWd+IVSd]³−[LVIDd]³) by Troy et al. (1972). Mice were humanely killed by cervical dislocation 1 week later. Blood was obtained immediately following by cardiac puncture and the heart, and other tissues were immediately removed. Hearts were washed in ice cold PBS and weighed prior to being snap frozen in liquid nitrogen.

Approximately 20 mg of ventricle tissue was homogenised in ice-cold lysis buffer (50 mM Tris pH7.5, 1 mM EDTA, 1 mM EGTA, 10% glycerol, 1% triton X-100, 50 mM NaF, 5 mM Na₄P₂O₇, 1 mM Na₃VO₄, 1 mM DTT, protease inhibitor cocktail) and protein content was determined using the BCA method. 20 pg of protein was separated by SDS-PAGE and transferred onto PVDF membrane using standard protocols. Blocked membranes were exposed to primary antibodies towards pro-caspase-1, mature caspase 1, total PKD, PKD phosphorylated at ser744 and 748 (Cell Signalling Technology, Danvers, USA) and α-tubulin (Sigma-Aldrich, St. Louis, USA). Membranes were visualised and bands quantified by using the ChemiDoc™ XRS+ with Image Lab™ software.

3D6 High Fat Diet (HFD) prevention study: Male C57BL6 mice were obtained from the Animal Resource Centre (Perth, Wash.) at 4 weeks of age and housed 4 mice per cage on a 12 hr light/dark cycle at a temperature of 22° C. and a constant humidity with a normal rodent diet. At 12 weeks of age, echocardiography was performed on all mice (n=24), to obtain pre-treatment measures of cardiac function, as follows. Mice were anaesthetised with inhalation of 1.5% isoflurane anaesthesia and echocardiography was performed using the Phillips HD15 diagnostic ultrasound system with a 15 MHz linear-array transducer by an experienced veterinarian. The velocity of blood flow through the mitral valve was analysed using Doppler mode imaging. These results were used to calculate the deceleration time and E:A ratio. Doppler imaging was also utilised to measure the velocity of blood flow through the aortic valve. The measurements were then used to calculate the ejection time, peak aortic flow and heart rate. M-mode imaging of the left ventricle was used to measure the thickness of the inter-ventricular septum (IVS), left ventricular internal diameter (LVID) and left ventricular posterior wall (LVPW) in both diastole (d) and end-systole (s), as well as systolic measures such as ejection fraction and fractional shortening. An estimation of LV mass was calculated from the m-mode imaging by using the formula (1.05[LVIDd+LVPWd+IVSd]³−[LVIDd]³) by Troy et al. (1972). All mice were then placed on a high fat diet (HFD) with 43% of calories from fat (23.5% by weight; SF04-001 High Fat Rodent Diet Based on D12451, Specialty Feeds, Glen Forrest, Wash.) for 13 weeks. At 12 weeks of age, mice were also administered 0.75 mg/kg bodyweight of either the Aβ₄₂ neutralising antibody 3D6 (#TAB-0809CLV, Creative Biolabs, Shirley, N.Y.) or the InVivo IgG2a Isotype Control antibody (#BE-0085, BioXCell, Lebanon, N.H.) weekly via intraperitoneal (i.p.) injection (n=12/group) for 13 weeks. Groups were selected based on fat mass, body weight and lean mass to match these variables as closely as possible between groups. Each cage contained 2 mice from each group.

After 10 weeks of the treatment period, mice underwent an oral glucose tolerance test (OGTT). Following a 5 hour fast, baseline readings of blood glucose were collected via a tail bleed of the mice using a hand-held glucometer (AccuCheck Performa). Mice were then administered 50 mg of glucose via oral gavage and blood glucose was measured 15, 30, 45, 60- and 90-minutes post administration. An additional 30 μL of blood was collected at baseline and 15, 30- and 60-minutes post administration in heparinised tubes for analysis of serum insulin concentration. Blood was centrifuged at 10,000 g for 10 minutes at 4° C. and plasma was collected by removing the supernatant. Plasma from the OGTT was analysed for insulin content using the Mouse Ultrasensitive Insulin ELISA (ALPCO, Salem, N.H.). An insulin tolerance test (ITT) 11 weeks into the treatment period. Following a 5 hour fast, baseline readings of blood glucose were collected via a tail bleed of mice using a hand-held glucometer (AccuCheck Performa). Mice were administered of humulin via i.p. injection and blood glucose was measured 20, 40, 60, 90- and 120-minutes post administration. Echocardiography was then performed 12 weeks into the treatment period, as described above, to obtain post-treatment measures of cardiac function. Changes in cardiac function parameters were expressed as a percentage of the baseline measure. Mice were sacrificed following 13 weeks of the treatment period. At the conclusion of the treatment period, mice were killed via cervical dislocation following a 5-hr fasting period. Blood was obtained immediately following by cardiac puncture and the heart, and other tissues were immediately removed. Hearts were washed in ice cold PBS and weighed prior to being snap frozen in liquid nitrogen.

Aβ₄₂ and mGluR5 NAM administration study: Lyophilised recombinant Aβ₄₂ (Millipore) and scrambled control peptide (ScrAβ₄₂; Millipore) were resuspended in 1% NH₄OH and aliquoted at 200 ng/ml in H₂O and stored at −80° C. for no longer than 4 weeks. Basimglurant (MedChemExpress) was resuspended in 100% DMSO before being diluted in a hydroxypropyl cellulose solution to give a final solution containing 0.25 mg/mL Basimglurant in 10% hydroxypropyl cellulose and 5% DMSO. A vehicle solution containing 10% hydroxypropyl cellulose and 5% DMSO was also made. Aliquots of both drug and vehicle were stored at −80° C. until required. Male C57BL6 mice were obtained from the Animal Resource Centre (Perth, Wash.) at 4 weeks of age and housed with 4 mice per cage on a 12 hr light/dark cycle at a temperature of 22° C. and a constant humidity with a normal rodent diet. At 12 weeks of age, mice were grouped according to body mass and composition, determined by EchoMRI. Mice were then administered with 1 mg/kg of Basimglurant or vehicle by oral gavage and approximately one hour later were administered 1 pg of recombinant Aβ₄₂ or ScrAβ₄₂ (n=12/group per cohort) by i.p. injection once/day for 5 wks. After 4 weeks of drug and peptide administration, cardiac function was assessed by echocardiography as follows. Mice were anaesthetised with inhalation of 1.5% isoflurane anaesthesia and echocardiography was performed using the Phillips HD15 diagnostic ultrasound system with a 15 MHz linear-array transducer by an experienced veterinarian. The velocity of blood flow through the mitral valve was analysed using Doppler mode imaging. These results were used to calculate the deceleration time, peak E wave and A wave velocities, the E:A ratio, isovolumetric relaxation time (IVRT) and isovolumetric contraction time (IVCT). Doppler imaging was also utilised to measure the velocity of blood flow through the aortic valve. The measurements were then used to calculate the ejection time (LVET), peak aortic flow and heart rate. M-mode imaging of the left ventricle was used to measure the thickness of the inter-ventricular septum (IVS), left ventricular internal diameter (LVID) and left ventricular posterior wall (LVPW) in both diastole (d) and end-systole (s) as well as systolic measures such as ejection fraction and fractional shortening. An estimation of LV mass was calculated from the m-mode imaging by using the formula (1.05[LVIDd+LVPWd+IVSd]³−[LVIDd]³) by Troy et al. (1972). The myocardial performance index (MPI), a global index of both diastolic and systolic cardiac function, was calculated using the formula ([IVCT+IVRT]/LVET), by Tei et al. (1995). Mice were humanely killed by cervical dislocation 1 week later. Blood was obtained immediately following by cardiac puncture and the heart, and other tissues were immediately removed. Hearts were washed in ice cold PBS and weighed prior to being snap frozen in liquid nitrogen. Plasma Aβ42 was measured using a high sensitivity ELISA kit (Wako Diagnostics) and plasma that was diluted 1:10 with assay buffer.

3D6-High Fat Diet (HFD) treatment study: At 12 weeks of age, echocardiography was performed on mice (n=36) to obtain baseline measures of cardiac function. Mice were then separated into 3 groups of 12, which included a chow/control, HFD/control and HFD/3D6 group. The groups were selected based on their measures of diastolic function, fat mass and bodyweight, to match these variables as closely as possible. The two HFD groups were then placed on a HFD with 43% of calories from fat (23.5% by weight; SF04-001 High Fat Rodent Diet Based on D12451, Specialty Feeds, Glen Forrest, Wash.) for 22 weeks, while the chow group remained on a standard chow diet. Following 15 weeks of the diet period, echocardiography was again performed on all groups to obtain pre-drug treatment measures of cardiac function. The chow/control and HFD/control groups were then administered 0.75 mg/kg bodyweight of the InVivo IgG2a Isotype Control antibody (#BE-0085, BioXCell, Lebanon, N.H.) weekly via I.P injection for 7 weeks while the HFD/3D6 group received 0.75 mg/kg bodyweight of the 3D6 antibody (#TAB-0809CLV, Creative Biolabs, Shirley, N.Y.). Echocardiography was then performed following 6 weeks of the treatment period to obtain post-drug treatment measures of cardiac function. Following 7 weeks of the drug administration, mice were humanely killed via cervical dislocation and blood was immediately obtained via cardiac puncture and stored in a heparinised tube. The heart, epididymal fat pad, mesenteric fat pad, liver, quadricep, hind limb and brain were then immediately dissected. The heart was 151 blotted prior to being weighed and all tissues were snap frozen in liquid nitrogen and stored at −80° C. Plasma Aβ42 was measured using a high sensitivity ELISA kit (Wako Diagnostics) and plasma that was diluted 1:10 with assay buffer. Cardiac TAG was measured using a triglyceride GPO-PAP kit (Roche Diagnostics) after extraction by KOH hydrolysis.

Aβ₄₀ administration study: Lyophilised recombinant Aβ₄₀ (Millipore) and scrambled control peptide (ScrAβ₄₀; Millipore) were resuspended in 1% NH₄OH and aliquoted at 200 ng/ml in H₂O and stored at −80° C. for no longer than 4 weeks. Male C57BL6 mice were obtained from the Animal Resource Centre (Perth, Wash.) at 4 weeks of age and housed with 4 mice per cage on a 12 hr light/dark cycle at a temperature of 22° C. and a constant humidity with a normal rodent diet. At 12 weeks of age, mice were grouped according to body mass and composition, determined by EchoMRI. Mice were then administered 1 μg of recombinant Aβ₄₀ or ScrAβ₄₀ (n=12/group per cohort) by i.p. injection once/day for 5 wks. After 4 weeks of peptide administration, cardiac function was assessed by echocardiography as follows. Mice were anaesthetised with inhalation of 1.5% isoflurane anaesthesia and echocardiography was performed using the Phillips HD15 diagnostic ultrasound system with a 15 MHz linear-array transducer by an experienced veterinarian. The velocity of blood flow through the mitral valve was analysed using Doppler mode imaging. These results were used to calculate the deceleration time and E:A ratio. Doppler imaging was also utilised to measure the velocity of blood flow through the aortic valve. The measurements were then used to calculate the ejection time, peak aortic flow and heart rate. M-mode imaging of the left ventricle was used to measure the thickness of the inter-ventricular septum (IVS), left ventricular internal diameter (LVID) and left ventricular posterior wall (LVPW) in both diastole (d) and end-systole (s) as well as systolic measures such as ejection fraction and fractional shortening. An estimation of LV mass was calculated from the m-mode imaging by using the formula (1.05 [LVIDd+LVPWd+IVSd]³−[LVIDd]³) by Troy et al. (1972). Mice were humanely killed by cervical dislocation 1 week later. Blood was obtained immediately following by cardiac puncture and the heart, and other tissues were immediately removed. Hearts were washed in ice cold PBS and weighed prior to being snap frozen in liquid nitrogen. Plasma 440 was measured using a high sensitivity ELISA kit (Wako Diagnostics) and plasma that was diluted 1:10 with assay buffer.

Neonatal ventricular cardiomyocyte experiments: Primary mouse neonatal ventricular cardiomyocytes were isolated from one day old neonate mice. For antagonist studies, cells were seeded in 12 well plates and left to recover for 24 hrs. Cells were then exposed to 125 nM Aβ42 for 24 hrs, and co-treated with vehicle, or antagonists towards various cell surface receptors and signalling pathways. At the conclusion of the treatment period, cells were harvested and RNA was isolated. Gene expression analyses were performed as described above. For metabolic analyses, cells were seeded in Seahorse V7 plates at a density of 100,000 cells per well in low glucose DMEM supplemented with 10% FBS. Cells were left to recover for 24 hr after the isolation before being infected with lentiviral vectors expressing either GFP (control), or constitutively active PKD. 48 hrs later, cells were assessed for metabolic flux parameters using a Seahorse XF24 analyser, including oxygen consumption rate (OCR) and extracellular acidification rate (ECAR) measures throughout sequential inhibition of mitochondrial respiratory chain complexes, and the dependency and capacity for the oxidation of the fatty acid palmitate. For western blotting analysis, cells were seeded into 6 well plates and infected with lentiviral vectors as described above. 48 hr after infection, cells were harvested and homogenized in protein lysis buffer, total protein concentration was determined and western blotting was performed, as described above. Membranes were exposed to primary antibodies towards total PKD, PKD phosphorylated at ser744/48 and ser 916, total HDAC5, HDAC5 phosphorylated at ser498, total Tcn1, Tcn1 phosphorylated at ser23/24, total CREB and CREB phosphorylated at ser 133 (Cell Signalling Technology), as well as GFP (Sigma).

Cardiac-specific dominant negative PKD mouse study: A novel dominant negative (DN) PKD, with a single point mutation in the ATP-binding domain (K612W) knock-in (at the Rosa26 locus) mouse regulated by a floxed STOP promoter was generated. This mouse expresses DN PKD only when crossed with Cre lines to excise the STOP codon from the CAG promoter. Mice heterozygous for the DN PKD knock-in were crossed with mice expressing estrogen-receptor chimeric Cre, driven by the aMHC promoter so that Cre is expressed exclusively in cardiomyocytes. This modified Cre only becomes nuclear and active against LoxP sites in the presence of tamoxifen. Through this breeding strategy, offspring are either heterozygous or null for the DN PKD knock-in and are all heterozygous for Cre. At 12 weeks of age, all mice were administered 40 mg/kg of tamoxifen via i.p. injection. Two weeks later, mice were fed either regular chow, or a high fat diet consisting of 43% fat for a period 12 weeks. At the conclusion of the diet period, mice underwent echocardiographic assessment of cardiac morphology and function, as described above.

Basimglurant-High Fat Diet (HFD) treatment study: At 12 weeks of age, mice (n=30) were placed on a HFD with 43% of calories from fat (23.5% by weight; SF04-001 High Fat Rodent Diet Based on D12451, Specialty Feeds, Glen Forrest, Wash.). After 13 weeks of the diet period, echocardiography was performed on all mice to obtain pre-drug treatment measures of cardiac function. Mice were allocated to treatment groups so that measures of cardiac function and morphology were matched as best as possible. Basimglurant (MedChemExpress) was resuspended in 100% DMSO before being diluted in a hydroxypropyl cellulose solution to give a final solution containing 0.25 mg/mL Basimglurant in 10% hydroxypropyl cellulose and 5% DMSO. A vehicle solution containing 10% hydroxypropyl cellulose and 5% DMSO was also made. Aliquots of both drug and vehicle were stored at −80° C. until required. The HFD/Basimglurant group was administered 1 mg/kg of Basimglurant per day by oral gavage, while the HFD/control group received an equivalent volume of vehicle. Echocardiography was then performed following 4 weeks of the treatment period to obtain post-drug treatment measures of cardiac function. Following 5 weeks of the drug administration, mice were humanely killed via cervical dislocation and blood was immediately obtained via cardiac puncture and stored in a heparinised tube. The heart, epididymal fat pad, mesenteric fat pad, liver, quadricep, hind limb and brain were then immediately dissected. The heart was 151 blotted prior to being weighed and all tissues were snap frozen in liquid nitrogen and stored at −80° C. Plasma Aβ42 was measured using a high sensitivity ELISA kit (Wako Diagnostics) and plasma that was diluted 1:10 with assay buffer.

### Example 2—Chronic Aβ42 Administration Alters Cardiac Metabolism

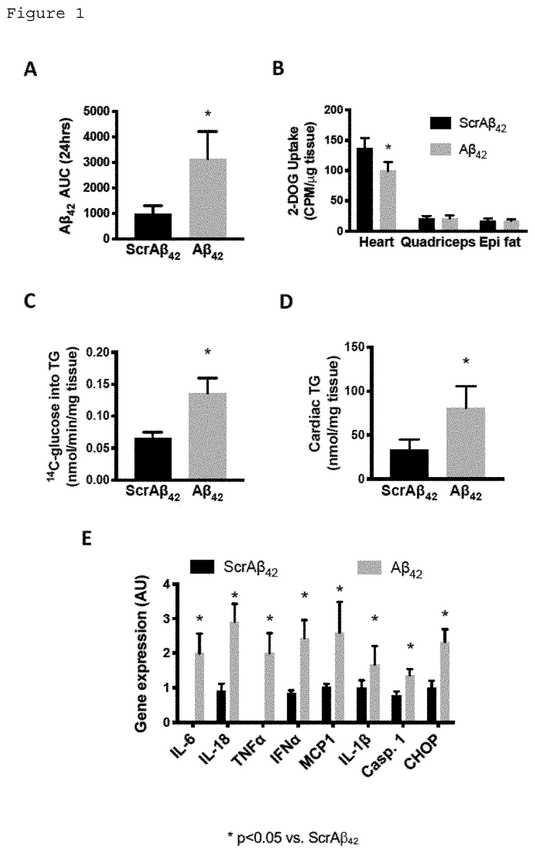

The in vivo effects of Aβ₄₂ were assessed by i.p. administration of 1 μg/day of Aβ₄₂, while control mice were administered a scrambled Aβ₄₂ peptide (ScrAβ₄₂) for a period of five weeks. Administration of Aβ₄₂ increased plasma Aβ₄₂ approximately 3-fold compared with administration of ScrAβ₄₂ (FIG. 1A) There was no change in body weight, body composition or food intake in mice administered Aβ₄₂. After five weeks of peptide administration, a GTT with glucose tracers was performed. There was no difference in whole body glucose tolerance or plasma insulin throughout the GTT between ScrAβ₄₂ or Aβ₄₂ administered mice. However, when tissues were assessed for glucose uptake throughout the GTT, by 2-DOG uptake, an ˜25% decrease in glucose uptake by the heart was observed in mice administered Aβ₄₂ (FIG. 1B). Glucose utilisation was further analysed using ¹⁴C-glucose labelling which revealed greater glucose incorporation into TAG (FIG. 1C) and increased total TAG (FIG. 1D) in Aβ₄₂ administered mice. This was associated with gene expression changes indicative of cardiac stress responses, including inflammation and endoplasmic reticulum stress (FIG. 1E).

### Example 3—Chronic Aβ42 Administration Alters Cardiac Function

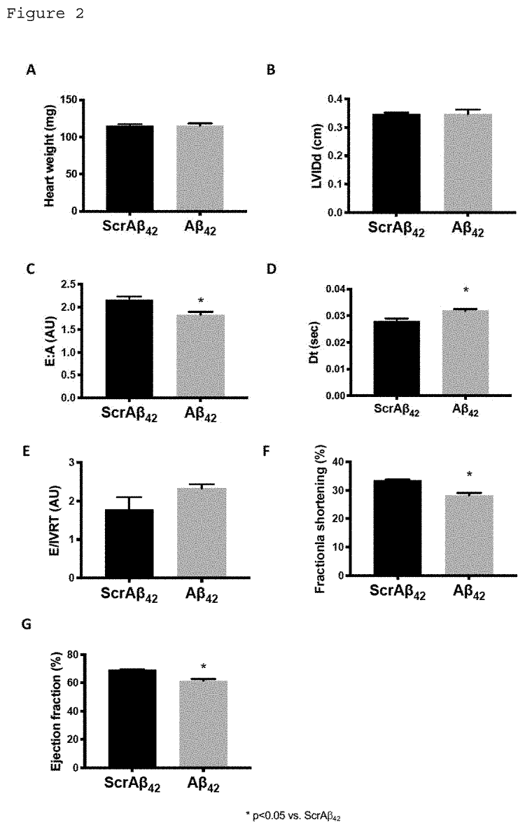

To assess whether Aβ₄₂ administration affected cardiac function, mice were administered ScrAβ₄₂ or Aβ₄₂ for five weeks prior to echocardiography. Hearts were also collected for morphological analysis (FIG. 2). Administration of Aβ₄₂ had no effect on gross heart weight (FIG. 2A) or of internal dimensions of the left ventricle (LVIDd; FIG. 2B). However, indices of diastolic dysfunction were evident in mice administered Aβ₄₂, including reduced E:A ratio (FIG. 2C) and increased deceleration time (FIG. 2D). There was no significant difference between groups when the peak blood flow velocity into the left ventricle during the relaxation phase in early dystole (E) was normalised by the relaxation time (isovolumetric relaxation time; IVRT) (FIG. 2E). This is a index of left artrial pressure and suggests that the diastolic dysfunction observed could be classified as grade 1. Furthermore, fractional shortening (FIG. 2F) and ejection fraction (FIG. 2G) were both reduced in Aβ₄₂ administered mice, which is indicative of systolic dysfunction.

### Example 4—Administration of Anti-Aβ42 Antibodies Preserves Diastolic Function in Development of Obesity

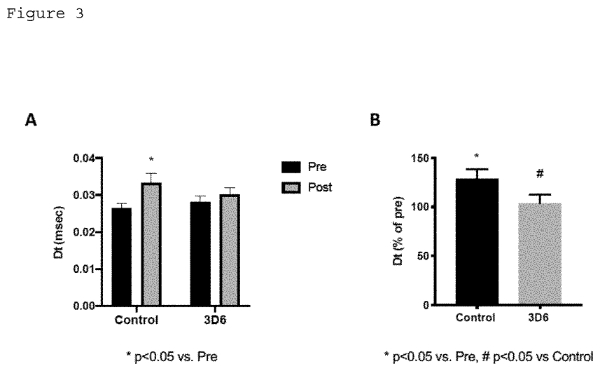

Echocardiography Doppler imaging of the mitral valve was used to assess the deceleration time, a critical measure of diastolic function (FIG. 3). Following 14 weeks of high fat feeding, mice administered the control antibody had an increase in deceleration time (FIG. 3A), indicating deterioration of diastolic function. In contrast, mice administered the 3D6 antibody showed either preserved or decreased deceleration time (FIG. 3A). Expressed relative to baseline measures, mice administered the control antibody had a statistically significant ˜30% increase in deceleration time (FIG. 3B), indicative of diastolic dysfunction. In contrast, deceleration time in mice administered the 3D6 antibody did not change from baseline levels (FIG. 3B). The relative change in deceleration time from baseline was significantly different between control and 3D6 antibody administered groups (FIG. 3B).

### Example 5—Administration of Anti-Aβ42 Antibodies Prevents Concentric Hypertrophy in Development of Obesity

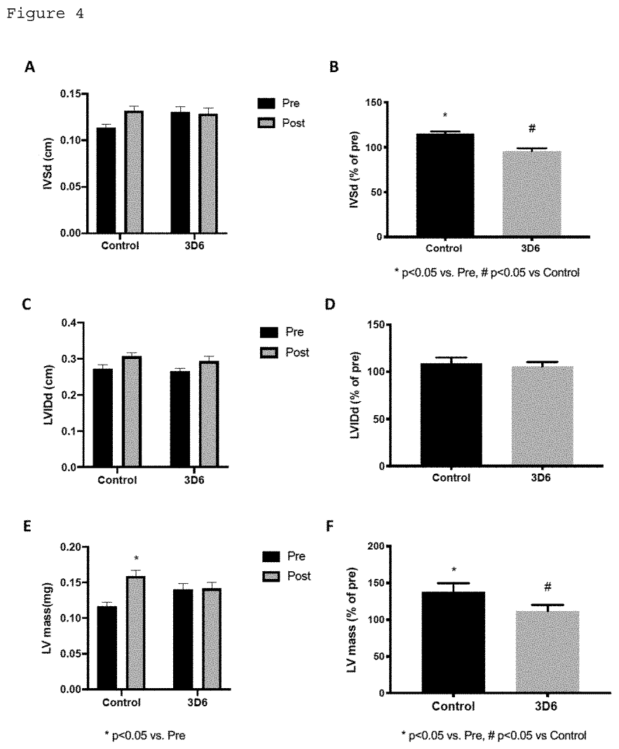

Echocardiographic M-mode imaging was used to characterise the morphology of the left ventricle (FIG. 4). Mice administered control antibody tended to have an increased intraventricular septum thickness at end-diastole (IVSd), a measure of hypertrophy, following the development of obesity, which was not observed in mice administered 3D6 antibody (FIG. 4A). Expressed relative to pre high fat diet values, IVSd significantly increased 115% in mice administered control antibody, while in mice administered 3D6 antibody this value was 95% (FIG. 4B). The relative change in IVSd from pre high fat diet values was significantly different between control and 3D6 antibody administered groups (FIG. 4B). There were no differences between the left ventricle internal diameter at end-diastole (LVIDd), a measure of left ventricle dilation, between groups (FIGS. 4C and D). However, mice administered control antibody significantly increased calculated left ventricular mass, a measure of hypertrophy, throughout the development of obesity, which was not observed in mice administered 3D6 antibody (FIG. 4E). Expressed relative to pre high fat diet values, left ventricular mass significantly increased 138% in mice administered control antibody (FIG. 4F). The relative change in left ventricular mass from pre high fat diet values was significantly different between control and 3D6 antibody administered groups (FIG. 4F).

### Example 6—Chronic Aβ42 Administration Primes and Activates the NLRP3 Inflammasome

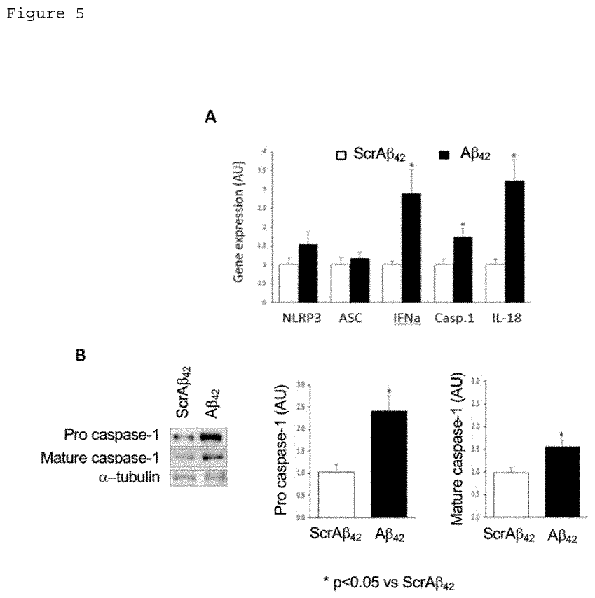

Chronic administration of Aβ42 to mice increased the expression of genes associated with priming of the NLRP3 inflammasome, including Nlrp3, Ifna and Il18 (FIG. 5A). Furthermore, chronic Aβ42 administration also increased indices of NLRP3 assembly, including increased pro-caspase 1 and increased mature caspase 1 (FIG. 5B).

### Example 7—Chronic Aβ42 Administration Activates PKD and is Important for the Aβ42-Mediated Inflammatory Response

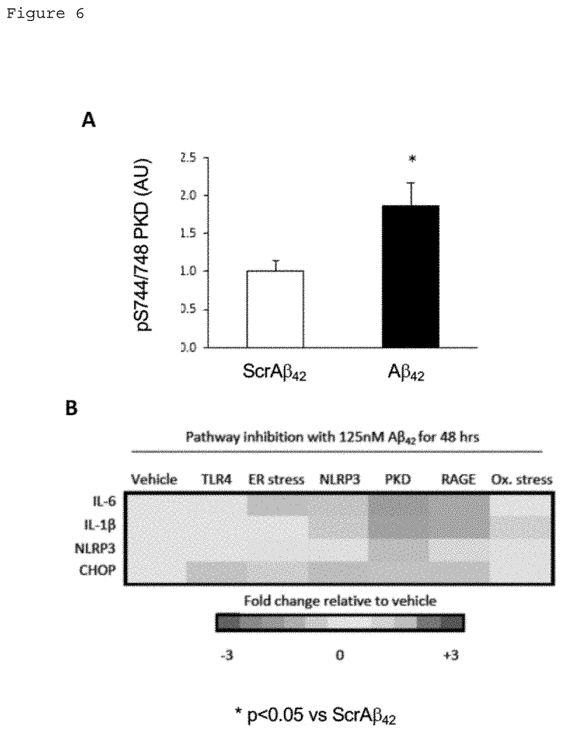

Chronic administration of Aβ42 to mice increased the phosphorylation of PKD at ser744 and 748, sites associated with its activation (FIG. 6A). In cardiomyocytes exposed to Aβ42, inhibition of PKD activity with 10 uM CID755673 prevented the Aβ42-induced increase in inflammatory gene expression (FIG. 6B).

### Example 8—PKD Regulates Cardiomyocyte Metabolism

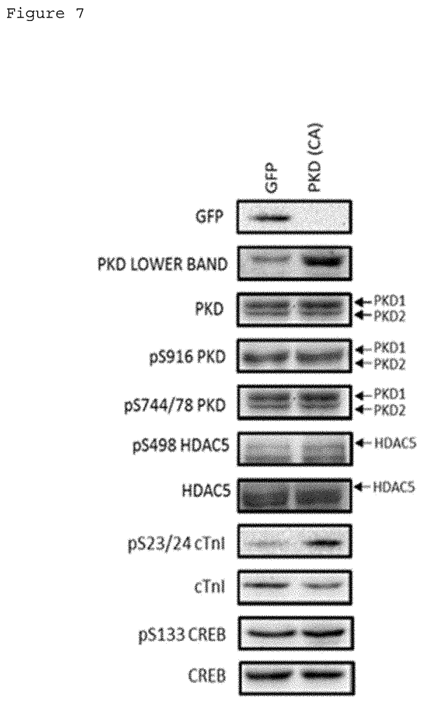

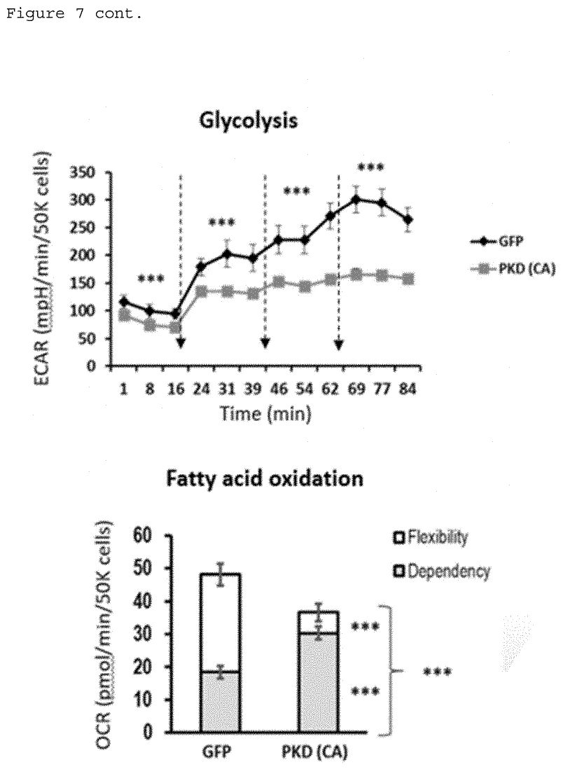

Cardiomyocytes over expressing constitutively active (CA) PKD (FIG. 7A) showed reduced glycolytic flux, represented by reduced SCAR, following the sequential addition of oligomycin (an ATP synthase inhibitor), FCCP (a mitochondrial uncoupler) and rotenone (a complex I inhibitor), suggesting reduced glucose utilisation (FIG. 7B). In contrast, these CA. PKD expressing cardiomyocytes had increased dependency on fatty acid oxidation, however their total capacity for fatty acid oxidation was reduced (FIG. 7C).

### Example 9—Inactivation of PKD Preserves Cardiac Function in Obesity

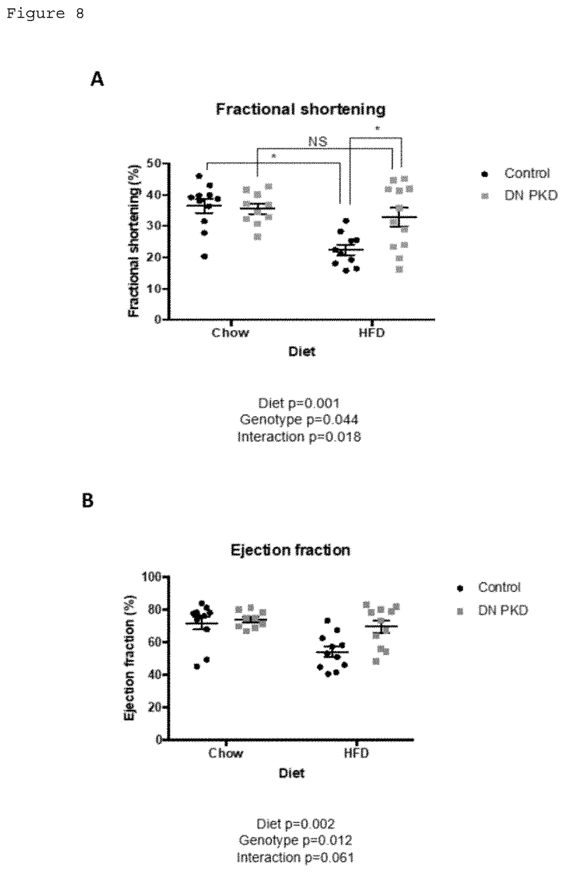

Cardiac function in control and cardiac-specific DN PKD mice fed either chow or a high fat diet for 12 weeks was assessed by echocardiography. This analysis showed that systolic function was preserved by inactivation of PKD in obesity. Control mice made obese through high fat feeding had a significant decrease in fractional shortening, which was not reduced by high fat feeding in cardiac-specific DN PKD mice (FIG. 8A). Furthermore, statistical main effects for both diet and genotype were observed for ejection fraction (FIG. 8B).

### Example 10—Preventing Decline in E Wave and Decline in Cardiac Performance Associated with Chronic Aβ42 Administration by Administration of mGluR5 NAM

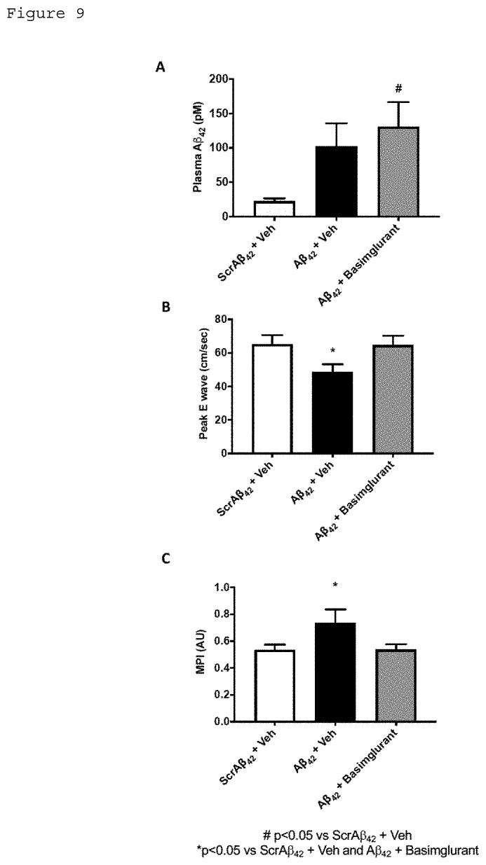

The effect of the mGluR5 NAM Basimglurant on Aβ42-induced cardiac dysfunction was examined. Plasma Aβ42 was elevated in both Aβ42+Veh and Aβ42+Basimglurant groups compared with the ScrAβ42+Veh group and was significantly increased in the Aβ42+Basimglurant relative to the ScrAβ42+Veh group (FIG. 9A). Peak E wave, a key index of diastolic function, was reduced in the Aβ42+Veh group, but not in the Aβ42+Basimglurant groups compared with the ScrAβ42+Veh group (FIG. 9B), indicating the Basimglurant preserves Aβ42-induced diastolic dysfunction. Similarly, the myocardial performance index (MPI), a unified measure of global cardiac function, was increased in the Aβ42+Veh group, but not in the Aβ42+Basimglurant groups compared with the ScrAβ42+Veh group, indicating the Basimglurant preserves Aβ42-induced cardiac dysfunction (FIG. 9C).

### Example 11—Administration of Anti-Aβ42 Antibodies Preserves Diastolic Function and Reduces Cardiac TAGs in Established Obesity

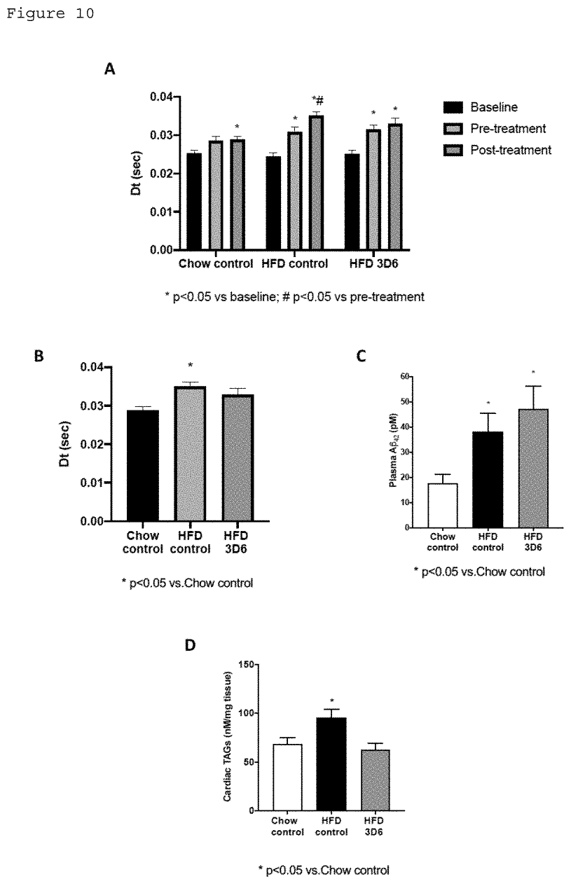

To assess the effect of treating obese mice with 3D6 on diastolic function, Doppler imaging of the mitral valve was conducting using echocardiography at the start of the study (Baseline), after 13 weeks of chow or HFD (Pre-treatment) and following 7 weeks of weekly 3D6 administration (Post-treatment) (FIG. 10).

In the chow control group, there was no significant change in DT between Baseline and Pre-treatment, but DT was significantly increased at Post-treatment compared with Baseline (FIG. 10A). In the HFD control group, DT significantly increased from Baseline to Pre-treatment and was further increased at Post-treatment (FIG. 10A). In contrast, the HFD 3D6 group showed a significant increase in DT between Baseline and Pre-treatment, however diastolic function did not deteriorate any further following 3D6 administration (FIG. 10A). When examining DT between groups at the conclusion of treatment period, DT was significantly elevated in the HFD control group compared to the Chow control group, while DT was not significantly different from Chow control in the HFD 3D6 group (FIG. 10B). The effect of the intervention on plasma Aβ42 was examined. In the HFD control group, Aβ42 levels were significantly increased compared with the Chow control group (FIG. 10C). Consistent with the neutralising function of the 3D6 antibody, plasma Aβ42 remained elevated in the HFD 3D6 group compared with Chow control (FIG. 10C). However, 3D6 treatment reduced cardiac TAG accumulation in obese mice (FIG. 10D).

### Example 12—Aβ40 Chronic Administration does not Alter Cardiac Function

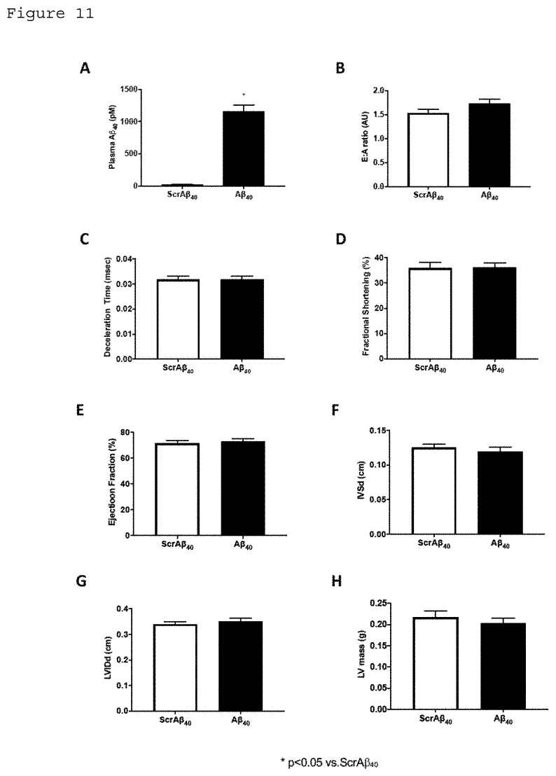

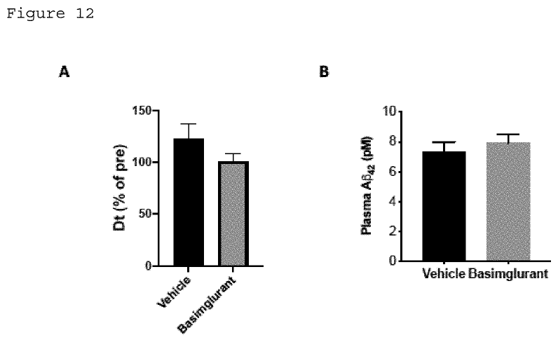

To determine whether other amyloid beta peptides could induce cardiac dysfunction similar to Aβ42, mice were administered Aβ40 or scrambled Aβ40 (ScrAβ40) at 1 pg/day by i.p. injection for 5 weeks, prior to echocardiography (FIG. 11). Administration of Aβ40 significantly increased plasma Aβ40 (FIG. 11A). However, administration of Aβ40 did not have any effect on indices of diastolic function, including E:A ratio (FIG. 11B) and DT (FIG. 11C), nor any effect on indices of systolic function, including fractional shortening (FIG. 11D) and ejection fraction (FIG. 11E). In addition, Aβ40 administration had no effect on cardiac morphology measures, including IVSd (FIG. 12F), LVIDd (FIG. 11G) and LV mass (FIG. 11H).

### Example 13—Administration of mGluR5 NAM Preserves Diastolic Function in Established Obesity

To assess the effect of treating obese mice with the mGluR5 NAM Basimglurant on diastolic function, Doppler imaging of the mitral valve was conducting using echocardiography after 13 weeks of HFD (Pre-treatment) and following a further 4 weeks of daily Basimglurant treatment (Post-treatment)(FIG. 12). When examining the % change in DT throughout the treatment period, DT was 122% of baseline levels after treatment in the vehicle group, while DT was 100% of baseline levels after treatment in the Basimglurant group (FIG. 12A). Consistent with the idea that Basimglurant acts as an Aβ₄₂ receptor antagonist, Basimglurant did not have any effect on plasma Aβ₄₂ levels (FIG. 12B). The logical extension of these data is that extended treatment with Basimglurant in obese mice slows the deterioration in DT in obesity with established cardiac dysfunction.

### Example 14—Discussion and Conclusion

These data indicate that Aβ42 alters cardiac metabolism and function and has particular impact on diastole. Without being bound by hypothesis, it is believed that the alteration or reprogramming of cardiac metabolism may arise from an Aβ42 mediated or associated inflammatory response.

Administration of Aβ42 to mice reduced cardiac glucose uptake and shunted glucose into TAG synthesis, leading to TAG accumulation. Reduced glucose uptake and utilisation increases the reliance on fatty acid oxidation, which reduces cardiac efficiency. This is due to the greater O₂ cost to produce ATP from beta oxidation, which impairs ATP production and results in impaired cardiac relaxation. This leads to impaired diastolic function because the diastolic relaxation phase has large energetic and ATP requirements, as Ca²⁺ reuptake and normalisation of membrane ion balances is ATP dependent. Further, the relaxation phase is much longer than systole. Hence the increased reliance on fatty acid oxidation leads to the observed diastolic dysfunction.

Reduced glucose uptake and TAG accumulation are phenotypic traits of cardiomyopathy associated with obesity, whereby altered cardiac metabolism leads to impaired relaxation of the heart, or diastolic dysfunction, which is sufficient to initiate progression to heart failure. Over time, this can lead to concentric hypertrophy and can often also present with impaired systolic function. Consistent with this, administration of Aβ42 to mice impaired both diastolic and systolic function. However, these effects on cardiac function were not observed in mice administered Aβ40, suggesting that the effects of amyloid β on the heart are restricted to the isoform of 42 amino acids.

It is believed that Aβ42 signalling through mGluR5 activates PKD in cardiomyocytes. Activation of PKD results in an inflammatory response and priming of the assembly of the NLRP3 inflammasome, and reprograms cardiomyocyte metabolism such that glucose utilisation is reduced and fatty acid utilisation is increased. These are hallmark features of the pathogenesis of LVDD in obesity. Furthermore, as genetic inactivation of PKD preserved cardiac function in obesity, it is believed that PKD is an essential signalling component of both Aβ42 and obesity induced cardiac dysfunction.

These data also indicate that inhibiting Aβ42 function can prevent the development of diastolic dysfunction in obesity and in other individuals having a higher than normal plasma amount of Aβ42 and protein comprising same. Administration of the 3D6 Aβ42 neutralising antibody to mice throughout high fat feeding prevented the decline in diastolic function and development of concentric hypertrophy, represented by changes in IVSd and left ventricle mass, without left ventricle dilation (LVIDd). Consistent with the idea that mGluR5 is important for the effects of Aβ42 on the heart, administration of the mGluR5 NAM Basimglurant prevented Aβ42-induced diastolic dysfunction, represented by reduced peak E wave velocity. Furthermore, Basimglurant prevented the Aβ42-induced impairment of overall cardiac performance, represented by increased MPI. These data therefore indicate that mGluR5 NAM could be used to prevent diastolic dysfunction and progression to heart failure in obesity and conditions in individuals having a higher than normal plasma amount of Aβ42. The data presented support this idea, where Basimglurant administration influenced the deterioration in deceleration time (DT).

1. A method for preventing or treating diastolic dysfunction in an individual comprising administering to an individual in need of said prevention or treatment a therapeutically effective amount of a mGluR5 negative allosteric modulator (NAM).

2. The method of claim 1 wherein the mGluR5 NAM is compound 1 (basimglurant), compound 2 (mavoglurant) or compound 3 (dipraglurant).

3. The method of claim 2 wherein the individual has diastolic dysfunction.

4. The method of claim 3 wherein the individual has decreased cardiac glucose uptake.

5. The method of claim 4 wherein the individual has increased glucose incorporation into triacyl glycerol (TAG) in cardiac tissue.

6. The method of claim 5 wherein the individual has increased total cardiac TAG.

7. The method of claim 1 wherein the individual has a reduced E:A ratio.

8. The method of claim 1 wherein the individual has an increased deceleration time.

9. The method of claim 1 wherein the individual has increased intra ventricular septal thickening.

10. The method of claim 1 wherein the individual has increased left ventricle (LV) mass.

11. The method of claim 1 wherein the individual has an elevated plasma amount of Aβ42.

12. The method of claim 1 wherein the mGluR5 NAM preserves or decreases E wave deceleration, thereby minimising diastolic dysfunction.

13. The method of claim 1 wherein the mGluR5 NAM prevents concentric hypertrophy.

14. The method of claim 1 wherein the mGluR5 NAM preserves or prevents intra-ventricular septal thickening.

15. The method of claim 1 wherein the mGluR5 NAM preserves LV mass or prevents increased LV mass.

16. The method of claim 1 wherein the individual is obese.

17. The method of claim 1 wherein the individual is pre-diabetic, or diabetic.

18. The method of claim 1 wherein the individual does not have Alzheimer's disease.

19. The method of claim 1 wherein the individual has been assessed to determine whether the individual has an elevated amount in plasma of Aβ42.

20. The method of claim 19 wherein the individual is provided with a therapeutically effective amount of mGluR5 NAM, where the individual has been assessed as having an elevated plasma amount of Aβ42.
